(19) United States
(12) Patent Application Publication
(10) Pub. No.: US 2023/0360725 A1
(43) Pub. Date: Nov. 9, 2023

(54) DETECTING DEGRADATION BASED ON STRAND BIAS

(71) Applicant: GUARDANT HEALTH, INC., Palo Alto, CA (US)

(72) Inventor: Zachary Scott BOHANNAN, Oakland, CA (US)

(21) Appl. No.: 18/314,736

(22) Filed: May 9, 2023

Related U.S. Application Data
(60) Provisional application No. 63/339,766, filed on May 9, 2022.

(51) Int. Cl. G16B 20/20; G16B 20/10; G16B 30/10

(52) U.S. Cl. CPC G16B 20/10; G16B 20/20; G16B 30/10

(57) ABSTRACT

During operation, a computer system may receive information corresponding to identified molecules of deoxyribonucleic acid (DNA) in a tissue sample. Then, the computer system may determine a symmetric normalized odds ratio, which corresponds to damage of the DNA, based at least in part on the information. Moreover, determining the symmetric normalized odds ratio may include: computing a first odds ratio; computing a second odds ratio, where a numerator and a denominator in the second odds ratio are reversed relative to the first odds ratio; summing the first odds ratio and the second odds ratio; and normalizing the summation. Next, the computer system may calculate a confidence metric of one or more of the molecules based at least in part on the symmetric normalized odds ratio and a threshold, wherein the confidence metric corresponds to a probability that the one or more molecules are identified correctly.

### CROSS REFERENCE TO RELATED APPLICATIONS

This application claims priority under 35 U.S.C. 119(e) to U.S. Provisional Application Serial No. 63/339,766, “Detecting Degradation Based on Strand Bias,” filed on May 9, 2022, the contents of which are herein incorporated by reference.

### FIELD

The described embodiments relate to techniques for assessing confidence in one or more identified molecules in a tissue sample, such as tissue biopsy sample. Notably, the described embodiments relate to techniques for detecting degradation of deoxyribonucleic acid (DNA) based at least in part on strand bias.

### BACKGROUND

Advances in genetic analysis is enabling improved diagnosis and treatment of diseases. Notably, the analysis of genetic markers (such as the patterns or sequences of nucleotides or the genotype) in DNA from a tissue sample can improve the detection of diseases (such as cancer), as well as determine classifications that allow personalized or individual-specific treatments (which is sometimes referred to as ‘precision medicine’).

However, accurate analysis of DNA is often complicated by degradation and/or contamination of tissue samples. For example, the wide-spread use of formalin-fixed and paraffin-embedded (FFPE) tissue samples typically confounds accurate detection of mutations, such as: single nucleotide variations (SNVs), copy number variations (CNVs), gene fusions, insertions and deletions (indels), transversions, translocations, and/or inversions. In particular, the DNA extracted from formalin-fixed and paraffin-embedded tissue samples is usually fragmented and/or contains sequence artifacts. Moreover, strand bias (which is a type of sequencing bias in which one DNA strand is favored over the other or in which there is a marked compositional difference in the DNA strands) is often increased for damaged or contaminated the DNA.

Because it can be difficult to distinguish the resulting artifacts from true mutations, damaged or contaminated DNA can lead to incorrect results, such as a false positive or a false negative (e.g., incorrectly detecting a cancer or missing a cancer when it is present). Incorrect results undermine confidence in tissue biopsies, and can result in unnecessary or untimely therapeutic interventions, patient suffering and increased patient mortality.

### SUMMARY

A computer system that detects damage of DNA from or associated with a tissue sample is described. This computer system includes: an interface circuit; a computation device (such as a processor, a graphics processing unit or GPU, etc.) that executes program instructions; and memory that stores the program instructions. During operation, the computer system receives information corresponding to identified molecules of the DNA in the tissue sample. Then, the computer system determines a symmetric normalized odds ratio based at least in part on the information, where the symmetric normalized odds ratio corresponds to damage of the DNA. Moreover, determining the symmetric normalized odds ratio includes: computing a first odds ratio; computing a second odds ratio, where a numerator and a denominator in the second odds ratio are reversed relative to the first odds ratio; summing the first odds ratio and the second odds ratio; and normalizing the summation. Next, the computer system calculates a confidence metric of one or more of the molecules based at least in part on the symmetric normalized odds ratio and a threshold, where the confidence metric corresponds to a probability that the one or more molecules are identified correctly.

Note that the DNA damage may be associated with formalin fixing and paraffin embedding of the tissue sample. For example, the DNA damage may include: oxidated degradation of guanine to 8-oxoguanine (oxoG), or formaldehyde-induced DNA and chromatin damage, where the formaldehyde-induced DNA and chromatin damage may include: deamination, depurination, or histone-DNA crosslinks. In some embodiments, the information includes DNA sequences that each correspond to a single strand of DNA from the tissue sample and/or the DNA damage is associated with strand bias.

Moreover, the computer system may call variants in the DNA based at least in part on the confidence metric. Furthermore, the computer system may filter out a subset of the call variants based at least in part on the confidence metric. For example, the subset may include false-positive variant calls in the call variants associated with the DNA damage or that are incorrectly labeled as contamination. Alternatively or additionally, the subset may include the variant calls associated with strand bias. Note that the variant calls may include CNVs and/or SNVs.

Additionally, the computer system may adjust one or more sonication parameters for subsequent sonication of the tissue sample based at least in part on the confidence metric. Note that the confidence metric may correspond to a level of DNA fragmentation.

In some embodiments, a given odds ratio in the first odds ratio and the second odds ratio may be computed based at least in part on: a number of occurrences of a first allele on a first strand in the DNA; a number of occurrences of the first allele on a second strand in the DNA; a number of occurrences of a second allele on the first strand in the DNA; and a number of occurrences of the second allele on the second strand in the DNA. Note that the first allele may have a majority allele frequency and the second allele has a minority allele frequency.

Note that the computer system may determine a quality metric of the tissue sample by aggregating multiple confidence metrics for the molecules in the tissue sample.

Another embodiment provides a computer for use, e.g., in the computer system.

Another embodiment provides a computer-readable storage medium for use with the computer or the computer system. When executed by the computer or the computer system, this computer-readable storage medium causes the computer or the computer system to perform at least some of the aforementioned operations.

Another embodiment provides a method, which may be performed by the computer or the computer system. This method includes at least some of the aforementioned operations.

In some embodiments, a computer system is provided, comprising: an interface circuit; a computation device coupled to the interface circuit; and memory, coupled to the computation device, configured to store program instructions, wherein, when executed by the computation device, the program instructions cause the computer system to perform one or more operations comprising: receiving information corresponding to molecules of deoxyribonucleic acid (DNA) from a tissue sample; determining a symmetric normalized odds ratio based at least in part on the information, wherein determining the symmetric normalized odds ratio comprises: computing a first odds ratio; computing a second odds ratio, wherein a numerator and a denominator in the second odds ratio are reversed relative to the first odds ratio; summing the first odds ratio and the second odds ratio; and normalizing the summation; and calculating a quality metric of the tissue sample based at least in part on the symmetric normalized odds ratio and a threshold, wherein the quality of metric corresponds to damage of the DNA.

In some embodiments, the present disclosure provides for a non-transitory computer-readable storage medium for use in conjunction with a computer system, the computer-readable storage medium configured to store program instructions that, when executed by the computer system, causes the computer system to perform one or more operations comprising: receiving information corresponding to molecules of deoxyribonucleic acid (DNA) from a tissue sample; determining a symmetric normalized odds ratio based at least in part on the information, wherein determining the symmetric normalized odds ratio comprises: computing a first odds ratio; computing a second odds ratio, wherein a numerator and a denominator in the second odds ratio are reversed relative to the first odds ratio; summing the first odds ratio and the second odds ratio; and normalizing the summation; and calculating a quality metric of the tissue sample based at least in part on the symmetric normalized odds ratio and a threshold, wherein the quality of metric corresponds to damage of the DNA.

A method for detecting damage of deoxyribonucleic acid (DNA) from a tissue sample, comprising: by a computer system: receiving information corresponding to molecules of deoxyribonucleic acid (DNA) in the tissue sample; determining a symmetric normalized odds ratio based at least in part on the information, wherein determining the symmetric normalized odds ratio comprises: computing a first odds ratio; computing a second odds ratio, wherein a numerator and a denominator in the second odds ratio are reversed relative to the first odds ratio; summing the first odds ratio and the second odds ratio; and normalizing the summation; and calculating a quality metric of the tissue sample based at least in part on the symmetric normalized odds ratio and a threshold, wherein the quality of metric corresponds to the damage of the DNA.

This Summary is provided for purposes of illustrating some exemplary embodiments, so as to provide a basic understanding of some aspects of the subject matter described herein. Accordingly, it will be appreciated that the above-described features are examples and should not be construed to narrow the scope or spirit of the subject matter described herein in any way. Other features, aspects, and advantages of the subject matter described herein will become apparent from the following Detailed Description, Figures, and Claims.

### BRIEF DESCRIPTION OF THE FIGURES

FIG. 1 is a block diagram illustrating an example of a computer system in accordance with an embodiment of the present disclosure.

FIG. 2 is a flow diagram illustrating an example of a method for detecting damage of deoxyribonucleic acid (DNA) from a tissue sample using a computer system in FIG. 1 in accordance with an embodiment of the present disclosure.

FIG. 3 is a drawing illustrating an example of communication between components in a computer system in FIG. 1 in accordance with an embodiment of the present disclosure.

FIG. 4 is a drawing illustrating an example of the symmetric normalized odds ratio and the threshold for tissue samples in accordance with an embodiment of the present disclosure.

FIG. 5 is a drawing illustrating an example of the symmetric normalized odds ratio and the threshold for tissue samples in accordance with an embodiment of the present disclosure.

FIG. 6 is a drawing illustrating an example of the minor allele frequency (MAF), the symmetric normalized odds ratio and the threshold for tissue samples in accordance with an embodiment of the present disclosure.

FIG. 7 is a drawing illustrating an example of an impact of a confidence metric on reviews of tissue samples in accordance with an embodiment of the present disclosure.

FIG. 8 is a drawing illustrating an example of an impact of a confidence metric on reviews of tissue samples in accordance with an embodiment of the present disclosure.

FIG. 9 is a block diagram illustrating an example of a computer in accordance with an embodiment of the present disclosure.

Note that like reference numerals refer to corresponding parts throughout the drawings. Moreover, multiple instances of the same part are designated by a common prefix separated from an instance number by a dash.

### DETAILED DESCRIPTION

A computer system (which may include one or more computers) that detects damage of DNA from or associated with a tissue sample is described. During operation, the computer system may receive information corresponding to identified molecules of the DNA (which are sometimes referred to as ‘variants’) in the tissue sample. Then, the computer system may determine a symmetric normalized odds ratio based at least in part on the information, where the symmetric normalized odds ratio corresponds to damage of the DNA. Moreover, determining the symmetric normalized odds ratio may include: computing a first odds ratio; computing a second odds ratio, where a numerator and a denominator in the second odds ratio are reversed relative to the first odds ratio; summing the first odds ratio and the second odds ratio; and normalizing the summation. Next, the computer system may calculate a confidence metric of one or more of the molecules based at least in part on the symmetric normalized odds ratio and a threshold, where the confidence metric corresponds to a probability that the one or more molecules are identified correctly. Note that the DNA damage may be associated with formalin fixing and paraffin embedding of the tissue sample and/or may be associated with strand bias.

By determining the confidence metric, these analysis techniques may reduce the time and effort needed to analyze tissue samples, and may reduce the incidence of incorrect results (such as false positives and false negatives) when analyzing tissue samples. In the process, the analysis technique may increase confidence in tissue biopsies. Moreover, the analysis techniques may facilitate early detection of disease (such as cancer), and may provide improved diagnosis, tracking of disease progression and treatment. Furthermore, the analysis techniques may enable further understanding of a variety of types of cancer, and may facilitate the development of new treatments or therapeutic interventions. Consequently, the analysis techniques may reduce unnecessary or untimely therapeutic interventions, patient suffering and patient mortality.

In the discussion that follows, a reference allele and an alternate allele are used as illustrative examples of the first allele and the second allele. However, in other embodiments, the analysis techniques may be used with more complicated alleles, such as alleles that are not binary.

Moreover, in the discussion that follows, the analysis techniques are used to determine confidence metrics for tissue samples that include or correspond to a wide variety of genetic molecules or information, including: DNA (such as double-stranded or single-stranded when there is information available to establish stand bias), cell-free nucleic acid, ribonucleic acid (RNA), epigenetic information, gene expression or transcriptional state information, protein information, etc. In the discussion that follows, DNA corresponding to at least a portion of an individual’s genome is used as an illustrative example.

Furthermore, in order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term

As used in this specification and the appended claims, the singular forms ‘a’, ‘an’, and ‘the’ include plural references unless the context clearly dictates otherwise. Thus, e.g., a reference to ‘a method’ includes one or more methods, and/or operations of the type described herein and/or which will become apparent to those persons of ordinary skill in the art upon reading this disclosure and so forth.

Moreover, ‘optional’ or ‘optionally’ means that the subsequently described event or circumstance may or may not occur, and that the description includes cases where said event or circumstance occurs and cases where it does not.

Furthermore, throughout the description and claims of this specification, the word ‘comprise’ and variations of the word, such as ‘comprising’ and ‘comprises,’ means ‘including but not limited to,’ and is not intended to exclude, for example, other components, integers or steps. ‘Exemplary’ means ‘an example of’ and is not intended to convey an indication of a preferred or ideal configuration. ‘Such as’ is not used in a restrictive sense, but for explanatory purposes.

It is also to be understood that the terminology used herein is for the purpose of describing particular embodiments only, and is not intended to be limiting. Furthermore, unless defined otherwise, all technical and scientific terms used herein have the same meaning as commonly understood by one of ordinary skill in the art to which this disclosure pertains. In describing and claiming the methods, computer-readable media, and systems, the following terminology, and grammatical variants thereof, will be used in accordance with the definitions set forth below.

About: As used herein, ‘about’ or ‘approximately’ as applied to one or more values or elements of interest, refers to a value or element that is similar to a stated reference value or element. In certain embodiments, the term ‘about’ or ‘approximately’ refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Adapter: As used herein, ‘adapter’ refers to a short nucleic acid (e.g., less than about 500 nucleotides, less than about 100 nucleotides, or less than about 50 nucleotides in length) that is typically at least partially double-stranded and used to link to either or both ends of a given sample nucleic acid molecule. Adapters can include nucleic acid primer binding sites to permit amplification of a nucleic acid molecule flanked by adapters at both ends, and/or a sequencing primer binding site, including primer binding sites for sequencing applications, such as various next-generation sequencing (NGS) applications. Adapters can also include binding sites for capture probes, such as an oligonucleotide attached to a flow cell support or the like. Adapters can also include a nucleic acid tag as described herein. Nucleic acid tags are typically positioned relative to amplification primer and sequencing primer binding sites, such that a nucleic acid tag is included in amplicons and sequence reads of a given nucleic acid molecule. The same or different adapters can be linked to the respective ends of a nucleic acid molecule. In some embodiments, an adapter of the same sequence is linked to the respective ends of the nucleic acid molecule except that the nucleic acid tag differs. In some embodiments, the adapter is a Y-shaped adapter in which one end is blunt ended or tailed as described herein, for joining to a nucleic acid molecule, which is also blunt ended or tailed with one or more complementary nucleotides. In still other example embodiments, an adapter is a bell-shaped adapter that includes a blunt or tailed end for joining to a nucleic acid molecule to be analyzed. Other examples of adapters include T-tailed and C-tailed adapters.

Amplify: As used herein, ‘amplify’ or ‘amplification’ in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable. Amplification of polynucleotides encompasses a variety of chemical and enzymatic processes.

Barcode: As used herein, ‘barcode’ or ‘molecular barcode’ in the context of nucleic acids refers to a nucleic acid molecule including a sequence that can serve as a molecular identifier. For example, individual ‘barcode’ sequences are typically added to each DNA fragment during next-generation sequencing library preparation so that each read can be identified and sorted before the final data analysis. In some embodiments, the one or more molecular barcodes is at least 2, at least 4, at least 5, at least 6, at least 8, at least 10, at least 15 or at least 20 nucleotides in length. In some embodiments, the polynucleotides of the sample are tagged with at least 5, at least 10, at least 15, at least 20, at least 50, at least 100, at least 500, at least 1000, at least 5000, at least 10,000, at least 50,000 or at least 100,000 different tags/molecular barcodes.

Cancer Type: As used herein, ‘cancer type’ refers to a type or subtype of cancer defined, e.g., by histopathology. Cancer type can be defined by any conventional criterion, such as on the basis of occurrence in a given tissue (e.g., blood cancers, central nervous system or CNS, brain cancers, lung cancers such as small cell and non-small cell, skin cancers, nose cancers, throat cancers, liver cancers, bone cancers, lymphomas, pancreatic cancers, bowel cancers, rectal cancers, thyroid cancers, bladder cancers, kidney cancers, mouth cancers, stomach cancers, breast cancers, prostate cancers, ovarian cancers, lung cancers, intestinal cancers, soft tissue cancers, neuroendocrine cancers, gastroesophageal cancers, head and neck cancers, gynecological cancers, colorectal cancers, urothelial cancers, solid state cancers, heterogeneous cancers, homogenous cancers, or another cancer type), unknown primary origin and the like, and/or of the same cell lineage (e.g., carcinoma, sarcoma, lymphoma, cholangiocarcinoma, leukemia, mesothelioma, melanoma, or glioblastoma), and/or cancers exhibiting cancer markers, such as: Her2, CA15-3, CA19-9, CA-125, CEA, AFP, PSA, HCG, hormone receptor and NMP-22. Cancers can also be classified by stage (e.g., stage 1, 2, 3, or 4) and whether of primary or secondary origin.

Cell-Free Nucleic Acid: As used herein, ‘cell-free nucleic acid’ refers to nucleic acids not contained within or otherwise bound to a cell or, in some embodiments, nucleic acids remaining in a sample following the removal of intact cells. Notably, ‘cell-free nucleic acid’ is ‘cell free’ at the point of isolation from a subject. Therefore, cell-free nucleic acid may not encompass or may be different from isolated cellular DNA. Cell-free nucleic acids can include, e.g., all non-encapsulated nucleic acids sourced from a bodily fluid (e.g., blood, plasma, serum, urine, cerebrospinal fluid or CSF, etc.) from a subject. Cell-free nucleic acids include DNA (cfDNA), RNA (cfRNA), and hybrids thereof, including genomic DNA, mitochondrial DNA, circulating DNA, siRNA, miRNA, circulating RNA (cRNA), tRNA, rRNA, small nucleolar RNA (snoRNA), Piwi-interacting RNA (piRNA), long non-coding RNA (long ncRNA), and/or fragments of any of these. Cell-free nucleic acids can be double-stranded, single-stranded, or a hybrid thereof. A cell-free nucleic acid can be released into bodily fluid through secretion or cell-death processes, e.g., cellular necrosis, apoptosis, or the like. Some cell-free nucleic acids are released into bodily fluid from cancer cells, e.g., circulating tumor DNA (ctDNA). Others are released from healthy cells. CtDNA can be non-encapsulated tumor-derived fragmented DNA. Another example of cell-free nucleic acids is fetal DNA circulating freely in the maternal blood stream, also called cell-free fetal DNA (cffDNA). A cell-free nucleic acid can have one or more epigenetic modifications, e.g., a cell-free nucleic acid can be (or a histone associated with the cell-free nucleic acid can be) acetylated, 5-methylated, ubiquitylated, phosphorylated, sumoylated, ribosylated, and/or citrullinated.

Cellular Nucleic Acids: As used herein, ‘cellular nucleic acids’ means nucleic acids that are disposed within one or more cells from which the nucleic acids have originated, at least at the point a sample is taken or collected from a subject, even if those nucleic acids are subsequently removed (e.g., via cell lysis) as part of a given analytical process.

Contamination of samples: As used herein, the terms ‘contamination’ or ‘contamination of samples’ refer to any chemical or digital contamination of one sample with another sample. Contamination can be due to a variety of sources, such as, but not limited to: physical carryover of liquids between samples (e.g., pipetting, automated liquid handling via sample preparation or sequencer systems, manipulating amplified material, etc.), demultiplexing artifacts (e.g., base call errors confounding sample indexes that have limited pairwise Hamming distance, insertion/deletion confounding sample indexes that have limited pairwise edit distance, etc.), formalin fixing and paraffin embedding of a tissue sample and/or reagent impurities (e.g., sample index oligonucleotides contaminated, through either carryover of synthesis errors, with oligonucleotides containing another sample index).

Degradation of samples: As used herein, the terms ‘degradation’, ‘damage’, ‘degradation of samples’ or ‘damage to samples’ refer to physical (such as fragmentation) or chemical changes in a sample from its initial state. Degradation or damage can be due to a variety of causes, such as, but not limited to: fragmentation (such as breaking of a strand or a chromosome into one or more pieces), fusing (such as fusing of two or more strands), missing material (such as at least a portion of a strand or a chromosome) and/or another type of degradation or damage. In some embodiments, DNA degradation or damage may be associated with formalin fixing and paraffin embedding of a tissue sample. For example, DNA damage or degradation may include: oxidated degradation of guanine to 8-oxoguanine and/or formaldehyde-induced DNA and chromatin damage (such as deamination, depurination, and/or histone-DNA crosslinks).

Deoxyribonucleic Acid or Ribonucleic Acid: As used herein, ‘deoxyribonucleic acid’ or ‘DNA’ refers to a natural or modified nucleotide which has a hydrogen group at the 2′-position of the sugar moiety. DNA typically includes a chain of nucleotides including four types of nucleotide bases; adenine (A), thymine (T), cytosine (C), and guanine (G). As used herein, ‘ribonucleic acid’ or ‘RNA’ refers to a natural or modified nucleotide which has a hydroxyl group at the 2′-position of the sugar moiety. RNA typically includes a chain of nucleotides including four types of nucleotide bases; A, uracil (U), G, and C. As used herein, the term ‘nucleotide’ refers to a natural nucleotide or a modified nucleotide. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G). When a first nucleic acid strand binds to a second nucleic acid strand made up of nucleotides that are complementary to those in the first strand, the two strands bind to form a double strand. As used herein, ‘nucleic acid sequencing data,’ ‘nucleic acid sequencing information,’ ‘sequence information,’ ‘nucleic acid sequence,’ ‘nucleotide sequence,’ ‘genomic sequence,’ ‘genetic sequence,’ ‘fragment sequence,’ or ‘nucleic acid sequencing read’ denotes any information or data that is indicative of the order and identity of the nucleotide bases (e.g., adenine, guanine, cytosine, and thymine or uracil) in a molecule (e.g., a whole genome, whole transcriptome, exome, oligonucleotide, polynucleotide, or fragment) of a nucleic acid such as DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, and electronic signature-based systems.

Germline Mutation: As used herein, the terms ‘germline mutation’ or ‘germline variation’ are used interchangeably and refer to an inherited mutation (or not one arising post-conception). Germline mutations may be the only mutations that can be passed on to the offspring and may be present in every somatic cell and germline cell in the offspring.

Indel: As used herein, ‘indel’ refers to a mutation that involves the insertion or deletion of nucleotides in the genome of a subject.

Minor Allele Frequency: As used herein, ‘minor allele frequency’ or ‘MAF’ refers to the frequency at which minor alleles (e.g., not the most common allele) occurs in a given population of nucleic acids, such as a sample obtained from a subject. Genetic variants at a low minor allele frequency typically have a relatively low frequency of presence in a sample.

Mutant Allele Fraction: As used herein, ‘mutant allele fraction’ or ‘mutation dose’ refers to the fraction of nucleic acid molecules harboring an allelic alteration or mutation at a given genomic position/locus in a given sample. The mutant allele fraction is generally expressed as a fraction or a percentage. For example, a mutant allele fraction of a somatic variant may be less than 0.15.

Mutation: As used herein, ‘mutation’ refers to a variation from a known reference sequence and includes mutations such as, e.g., single nucleotide variants or SNVs, and insertions or deletions or indels. A mutation can be a germline or somatic mutation. In some embodiments, a reference sequence for purposes of comparison is a wildtype genomic sequence of the species of the subject providing a test sample, typically the human genome.

Neoplasm: As used herein, the terms ‘neoplasm’ and ‘tumor’ are used interchangeably. They refer to abnormal growth of cells in a subject. A neoplasm or tumor can be benign, potentially malignant, or malignant. A malignant tumor is a referred to as a cancer or a cancerous tumor.

Next Generation Sequencing: As used herein, ‘next generation sequencing’ or ‘NGS’ refers to sequencing technologies having increased throughput as compared to traditional Sanger- and capillary electrophoresis-based approaches, e.g., with the ability to generate hundreds of thousands of relatively small sequence reads at a time. Some examples of next generation sequencing techniques include, but are not limited to, sequencing by synthesis, sequencing by ligation, and sequencing by hybridization.

Nucleic Acid Tag: As used herein, ‘nucleic acid tag’ refers to a short nucleic acid (e.g., less than about 500 nucleotides, about 100 nucleotides, about 50 nucleotides, or about 10 nucleotides in length), used to distinguish nucleic acids from different samples (e.g., representing a sample index), or different nucleic acid molecules in the same sample (e.g., representing a molecular barcode), of different types, or which have undergone different processing. The nucleic acid tag includes a predetermined, fixed, non-random, random or semi-random oligonucleotide sequence. Such nucleic acid tags may be used to label different nucleic acid molecules or different nucleic acid samples or sub-samples. Nucleic acid tags can be single-stranded, double-stranded, or at least partially double-stranded. Nucleic acid tags optionally have the same length or varied lengths. Nucleic acid tags can also include double-stranded molecules having one or more blunt-ends, include 5′ or 3′ single-stranded regions (e.g., an overhang), and/or include one or more other single-stranded regions at other locations within a given molecule. Nucleic acid tags can be attached to one end or to both ends of the other nucleic acids (e.g., sample nucleic acids to be amplified and/or sequenced). Nucleic acid tags can be decoded to reveal information such as the sample of origin, form, or processing of a given nucleic acid. For example, nucleic acid tags can also be used to enable pooling and/or parallel processing of multiple samples including nucleic acids bearing different molecular barcodes and/or sample indexes in which the nucleic acids are subsequently being deconvolved by detecting (e.g., reading) the nucleic acid tags. Nucleic acid tags can also be referred to as identifiers (e.g., molecular identifier or sample identifier). Additionally, or alternatively, nucleic acid tags can be used as molecular barcodes (e.g., to distinguish between different molecules or amplicons of different parent molecules in the same sample or sub-sample). This includes, e.g., uniquely tagging different nucleic acid molecules in a given sample, or non-uniquely tagging such molecules. In the case of non-unique tagging applications, a limited number of tags (such as molecular barcodes) may be used to tag the nucleic acid molecules such that different molecules can be distinguished based on their endogenous sequence information (for example, start and/or stop positions where they map to a selected reference genome, a sub-sequence of one or both ends of a sequence, and/or length of a sequence) in combination with at least one molecular barcode. Typically, a sufficient number of different molecular barcodes are used such that there is a low probability (e.g., less than about a 10%, less than about a 5%, less than about a 1%, or less than about a 0.1% chance) that any two molecules may have the same endogenous sequence information (e.g., start and/or stop positions, subsequences of one or both ends of a sequence, and/or lengths) and also have the same molecular barcode.

Odds Ratio: As used herein, the term ‘odds ratio’ refers to a statistic that quantifies the strength of the association between two events, A and B. The odds ratio may be defined as the ratio of the odds or probability of A in the presence of B and the odds or probability of A in the absence of B, or equivalently (because of symmetry), the ratio of the odds or probability of B in the presence of A and the odds or probability of B in the absence of A. Two events are independent when the odds ratio equals 1, or the odds of one event are the same in either the presence or absence of the other event. If the odds ratio is greater than 1, then A and B are associated or related in the sense that, compared to the absence of B, the presence of B raises the odds of A, and symmetrically the presence of A raises the odds of B. Conversely, if the odds ratio is less than 1, then A and B are negatively related, and the presence of one event reduces the odds of the other event. As described further below, in some embodiments, an odds ratio may be a symmetric normalized odds ratio.

Polynucleotide: As used herein, ‘polynucleotide,’ ‘nucleic acid,’ ‘nucleic acid molecule,’ or ‘oligonucleotide’ refers to a linear polymer of nucleosides (including deoxyribonucleosides, ribonucleosides, or analogs thereof) joined by inter-nucleosidic linkages. Typically, a polynucleotide includes at least three nucleosides. Oligonucleotides often range in size from a few monomeric units, e.g., 3-4, to hundreds of monomeric units. Whenever a polynucleotide is represented by a sequence of letters, such as ‘ATGCCTG,’ it will be understood that the nucleotides are in 5′→3′ order from left to right and that in the case of DNA, ‘A’ denotes deoxyadenosine, ‘C’ denotes deoxycytidine, ‘G’ denotes deoxyguanosine, and ‘T’ denotes deoxythymidine, unless otherwise noted. The letters A, C, G, and T may be used to refer to the bases themselves, to nucleosides, or to nucleotides including the bases, as is standard in the art.

Reference Sequence: As used herein, ‘reference sequence’ refers to a known sequence used for purposes of comparison with experimentally determined sequences. For example, a known sequence can be an entire genome, a chromosome, or any segment thereof. A reference typically includes at least about 20, at least about 50, at least about 100, at least about 200, at least about 250, at least about 300, at least about 350, at least about 400, at least about 450, at least about 500, at least about 1000, or more than 1000 nucleotides. A reference sequence can align with a single contiguous sequence of a genome or chromosome or can include non-contiguous segments that align with different regions of a genome or chromosome. Examples of reference sequences include, e.g., human genomes, such as, hG19 and hG38.

Sample: As used herein, ‘sample’ means anything capable of being analyzed by the methods and/or systems disclosed herein. For example, a sample may include a normal tissue sample or a tissue sample associated with a type of disease, such as a type of cancer.

Sequencing: As used herein, ‘sequencing’ refers to any of a number of technologies used to determine the sequence (e.g., the identity and order of monomer units) of a biomolecule, e.g., a nucleic acid such as DNA or RNA. Examples of sequencing methods include, but are not limited to, targeted sequencing, single molecule real-time sequencing, exon or exome sequencing, intron sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, capillary electrophoresis, duplex sequencing, cycle sequencing, single-base extension sequencing, solid-phase sequencing, high-throughput sequencing, massively parallel signature sequencing, emulsion polymerase chain reaction (PCR), co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing (from Illumina of San Diego, California), SOLiD™ sequencing (from Life Technologies, a division of Thermo Fisher Scientific of Waltham, Massachusetts), MS-PET sequencing, and a combination thereof. In some embodiments, sequencing can be performer by a gene analyzer such as, e.g., gene analyzers commercially available from Illumina, Inc., Pacific Biosciences, Inc. (of Menlo Park, California), or Applied Biosystems/Thermo Fisher Scientific, among many others. Note that, in some embodiments, sequencing may include determining a base identity at a single position or loci.

Sequence Information: As used herein, ‘sequence information’ in the context of a nucleic acid polymer means the order and identity of monomer units (e.g., nucleotides, etc.) in that polymer.

Single Nucleotide Polymorphism: As used herein, the terms ‘single nucleotide polymorphism’ or ‘SNP’ are used interchangeably. They refer to a variation in a single nucleotide that occurs at a specific position in the genome, where each variation is present to some appreciable degree of frequency within a population (e.g., greater than about 1%).

Single Nucleotide Variant: As used herein, ‘single nucleotide variant’ or ‘SNV’ means a mutation or variation in a single nucleotide that occurs at a specific position in the genome.

Somatic Mutation: As used herein, the terms ‘somatic mutation’ or ‘somatic variation’ are used interchangeably. They refer to a mutation in the genome that occurs after conception. Somatic mutations can occur in any cell of the body except germ cells and accordingly, are not passed on to progeny.

Strand Bias: As used herein, the term ‘strand bias’ refers to a type of sequencing bias in which one DNA strand is favored over the other or in which there is a marked compositional difference in the DNA strands in a chromosome. Notably, in some sequencing techniques (such as high-throughput short-read sequencing), strand bias occurs when the genotype inferred from the positive or forward strand and the negative or reverse strand is significantly different. For example, at a given position in the genome, the reads mapped to the forward strand may support a heterozygous genotype, while the reads mapped to the reverse strand may support a homozygous genotype. More generally, strand bias occurs when there is a significant difference in the composition in the DNA strands in a chromosome, which may result in an incorrect assessment of the evidence for one allele versus another (such as a majority and a minority allele).

Subject: As used herein, ‘subject’ refers to an animal, such as a mammalian species (e.g., human) or avian (e.g., bird) species, or other organism, such as a plant. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or a predisposition to the disease, or an individual in need of therapy or suspected of needing therapy. The terms ‘individual’ or ‘patient’ are intended to be interchangeable with ‘subject.’

For example, a subject can be an individual who has been diagnosed with having a cancer, is going to receive a cancer therapy, and/or has received at least one cancer therapy. The subject can be in remission of a cancer. As another example, the subject can be an individual who is diagnosed of having an autoimmune disease. As another example, the subject can be a female individual who is pregnant or who is planning on getting pregnant, who may have been diagnosed of or suspected of having a disease, e.g., a cancer, an auto-immune disease.

Substantially identical: As used herein, the term ‘substantially identical’ refers to two different entities that are 99.9% identical, at least 95% identical, at least 90% identical, at least 85% identical, at least 80% identical, at least 75% identical, at least 70% identical, at least 60% identical or at least 50% identical. In cases where the entity is the molecular barcode, then the term ‘substantially identical’ refers to two different molecular barcodes that have a Hamming distance or edit distance of less than 2, less than 3, less than 4, less than 5, less than 6, less than 7 or less than 8. In cases where the entity is the beginning region or end region, then the term ‘substantially identical’ refers to two different regions that are within 1 bp, within 2 bp, within 3 bp, within 4 bp, within 5 bp, within 6 bp, within 7 bp, within 8 bp, within 9 bp, within 10 bp, within 11 bp, within 15 bp, within 20 bp or within 25 bp. In cases where the entity is the length of the polynucleotide, then the term ‘substantially identical’ refers to two different lengths that are within 1 bp, within 2 bp, within 3 bp, within 4 bp, within 5 bp, within 6 bp, within 7 bp, within 8 bp, within 9 bp, within 10 bp, within 11 bp, within 15 bp, within 20 bp, within 25 bp, within 30 bp, within 40 bp or within 50 bp.

Threshold: As used herein, ‘threshold’ refers to a predetermined value used to characterize experimentally determined values of the same parameter for different samples depending on their relation to the threshold. For example, the threshold for the p-value can refer to any predetermined value between 0 and 1 and is used to identify the origin of a nucleic acid variant.

Variant: As used herein, a ‘variant’ can be referred to as an allele. A variant is usually presented at a frequency of 50% (0.5) or 100% (1), depending on whether the allele is heterozygous or homozygous. For example, germline variants are inherited and usually have a frequency of 0.5 or 1. Somatic variants, however, are acquired variants and usually have a frequency of less than about 0.5. Major and minor alleles of a genetic locus refer to nucleic acids harboring the locus in which the locus is occupied by a nucleotide of a reference sequence, and a variant nucleotide different than the reference sequence respectively. Measurements at a locus can take the form of allelic fractions (Afs), which measure the frequency with which an allele is observed in a sample.

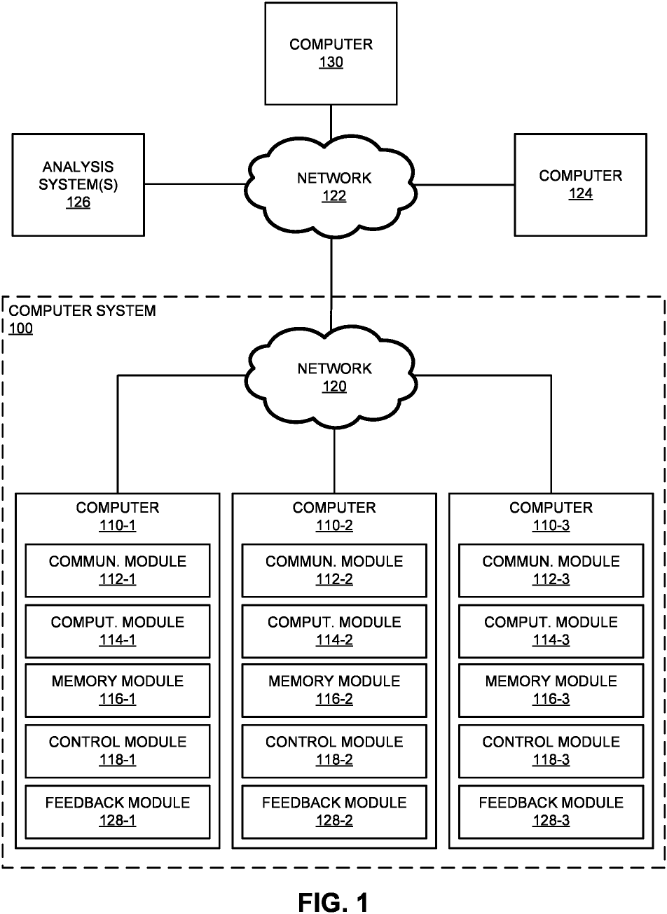

We now describe embodiments of the analysis techniques. FIG. 1 presents a block diagram illustrating an example of a computer system 100. This computer system may include one or more computers 110. These computers may include: communication modules 112, computation modules 114, memory modules 116, and optional control modules 118. Note that a given module or engine may be implemented in hardware and/or in software.

Communication modules 112 may communicate frames or packets with data or information (such as measurement results or control instructions) between computers 110 via a network 120 (such as the Internet and/or an intranet). For example, this communication may use a wired communication protocol, such as an Institute of Electrical and Electronics Engineers (IEEE) 802.3 standard (which is sometimes referred to as ‘Ethernet’) and/or another type of wired interface. Alternatively or additionally, communication modules 112 may communicate the data or the information using a wireless communication protocol, such as: an IEEE 802.11 standard (which is sometimes referred to as ‘Wi-Fi’, from the Wi-Fi Alliance of Austin, Texas), Bluetooth (from the Bluetooth Special Interest Group of Kirkland, Washington), a third generation or 3G communication protocol, a fourth generation or 4G communication protocol, e.g., Long Term Evolution or LTE (from the 3^{rd} Generation Partnership Project of Sophia Antipolis, Valbonne, France), LTE Advanced (LTE-A), a fifth generation or 5G communication protocol, other present or future developed advanced cellular communication protocol, or another type of wireless interface. For example, an IEEE 802.11 standard may include one or more of: IEEE 802.11a, IEEE 802.11b, IEEE 802.11g, IEEE 802.11-2007, IEEE 802.11n, IEEE 802.11-2012, IEEE 802.11-2016, IEEE 802.11ac, IEEE 802.11ax, IEEE 802.11ba, IEEE 802.11be, or other present or future developed IEEE 802.11 technologies.

In the described embodiments, processing a packet or a frame in a given one of computers 110 (such as computer 110-1) may include: receiving the signals with a packet or the frame; decoding/extracting the packet or the frame from the received signals to acquire the packet or the frame; and processing the packet or the frame to determine information contained in the payload of the packet or the frame. Note that the communication in FIG. 1 may be characterized by a variety of performance metrics, such as: a data rate for successful communication (which is sometimes referred to as ‘throughput’), an error rate (such as a retry or resend rate), a mean squared error of equalized signals relative to an equalization target, intersymbol interference, multipath interference, a signal-to-noise ratio, a width of an eye pattern, a ratio of number of bytes successfully communicated during a time interval (such as 1-10 s) to an estimated maximum number of bytes that can be communicated in the time interval (the latter of which is sometimes referred to as the ‘capacity’ of a communication channel or link), and/or a ratio of an actual data rate to an estimated data rate (which is sometimes referred to as ‘utilization’). Note that wireless communication between components in FIG. 1 uses one or more bands of frequencies, such as: 900 MHz, 2.4 GHz, 5 GHz, 6 GHz, 60 GHz, the Citizens Broadband Radio Spectrum or CBRS (e.g., a frequency band near 3.5 GHz), and/or a band of frequencies used by LTE or another cellular-telephone communication protocol or a data communication protocol. In some embodiments, the communication between the components may use multi-user transmission (such as orthogonal frequency division multiple access or OFDMA) and/or multiple-input multiple-output (MIMO).

Moreover, computation modules 114 may perform calculations using: one or more microprocessors, ASICs, microcontrollers, programmable-logic devices, GPUs and/or one or more digital signal processors (DSPs). Note that a given computation component is sometimes referred to as a ‘computation device’.

Furthermore, memory modules 116 may access stored data or information in memory that local in computer system 100 and/or that is remotely located from computer system 100. Notably, in some embodiments, one or more of memory modules 116 may access stored measurement results in the local memory, such as MRI data for one or more individuals (which, for multiple individuals, may include cases and controls or disease and healthy populations). Alternatively or additionally, in other embodiments, one or more memory modules 116 may access, via one or more of communication modules 112, stored measurement results in the remote memory in computer 124, e.g., via network 120 and network 122. Note that network 122 may include: the Internet and/or an intranet. In some embodiments, the measurement results are received from one or more analysis systems 126 (such as PCR, a whole genome sequencer or a partial genome sequencer, e.g., a whole exome sequencer or, more generally, a gene sequencer that uses: a gene sequencing panel, Sanger sequencing, capillary electrophoresis and fragment analysis, microarrays, ligation-based systems, polymerase-based systems, hybridization-based systems, direct or indirect nucleotide identification systems, pyrosequencing, ion- or pH-based detection systems, electronic signature-based systems, next generation sequencing, long-read genetic sequencing, sequencing based on nanopore technology, and/or another sequencing technique) via network 120 and network 122 and one or more of communication modules 112. Thus, in some embodiments at least some of the measurement results may have been received previously and may be stored in memory, while in other embodiments at least some of the measurement results may be received in real-time from the one or more analysis systems 126.

While FIG. 1 illustrates computer system 100 at a particular location, in other embodiments at least a portion of computer system 100 is implemented at more than one location. Thus, in some embodiments, computer system 100 is implemented in a centralized manner, while in other embodiments at least a portion of computer system 100 is implemented in a distributed manner (such as using cloud-computing resources). For example, in some embodiments, the one or more analysis systems 126 may include local hardware and/or software that performs at least some of the operations in the analysis techniques. This remote processing may reduce the amount of data that is communicated via network 120 and network 122. In addition, the remote processing may anonymize the measurement results that are communicated to and analyzed by computer system 100. This capability may help ensure computer system 100 is compatible and compliant with regulations, such as the Health Insurance Portability and Accountability Act, e.g., by removing or obfuscating protected health information in the measurement results.

Although we describe the computation environment shown in FIG. 1 as an example, in alternative embodiments, different numbers or types of components may be present in computer system 100. For example, some embodiments may include more or fewer components, a different component, and/or components may be combined into a single component, and/or a single component may be divided into two or more components.

As discussed previously, DNA damage can complicate analysis of DNA in samples, such as tissue biopsy samples. Notably, DNA damage may lead to incorrect analysis results, such as a false positive or a false negative. In turn, incorrect analysis results may cause an incorrect diagnosis or may result is delayed or incorrect treatment.

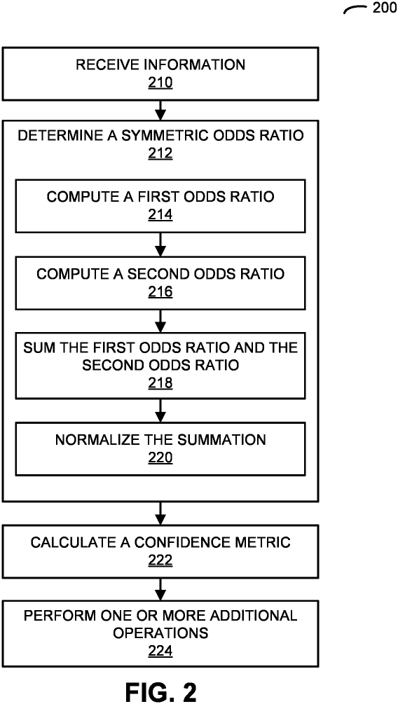

Moreover, as described further below with reference to FIGS. 2-8, in order to address these challenges computer system 100 may perform the analysis techniques. Notably, during the analysis techniques, one or more of optional control modules 118 may divide the analysis among computers 110. Then, a given computer (such as computer 110-1) may perform at least a designated portion of the analysis. In particular, computation module 114-1 may receive (e.g., access) information (e.g., using memory module 116-1) specifying identified genetic molecules (such as at least portions of DNA) from a tissue sample that is associated with a tissue biopsy. For example, the information may include or may be associated with histology. The information may include genotype information, such as: nucleotides as a function of location on at least a strand or in the DNA; mutations or variants as a function of location on at least a strand or in the DNA (such as an SNV, a CNV, a fusion, an insertion, a deletion and/or an epigenetic change); alleles as a function of location on at least a strand or in the DNA; epigenetic information as a function of on at least a strand or in the DNA; genetic information corresponding to molecules of DNA; and/or another type of genomic information as a function of location on at least a strand or in the DNA. Note that the aforementioned locations may be at least a subset of the loci in the DNA. Thus, the locations may include one or more loci in the DNA.

Then, computation module 114-1 may perform operations in the analysis techniques. Notably, the analysis techniques may include: determining a symmetric normalized odds ratio based at least in part on the information, where the symmetric normalized odds ratio corresponds to damage of the DNA. The symmetric normalized odds ratio may be determined by: computing a first odds ratio using the information; computing a second odds ratio using the information, where a numerator and a denominator in the second odds ratio are reversed relative to the first odds ratio (thus, the second odds ratio may be the inverse of the first odds ratio); summing the first odds ratio and the second odds ratio; and normalizing the summation. Next, the analysis techniques may include calculating a confidence metric of one or more of the molecules based at least in part on the symmetric normalized odds ratio and a threshold, where the confidence metric corresponds to a probability that the one or more molecules are identified correctly.

Note that the confidence metric may be effective in distinguishing biological signals from technical noise (such as sequencer/preservation error). Moreover, as noted previously, the confidence metric may correspond to a probability that the one or more molecules or biological variants are identified correctly (or accurately distinguished from variants caused by technical artifacts or sample degradation).

In some embodiments, a given odds ratio in the first odds ratio and the second odds ratio may be computed based at least in part on: a number of occurrences of a reference allele on a first strand in the DNA; a number of occurrences of the reference allele on a second strand in the DNA; a number of occurrences of a alternate allele on the first strand in the DNA; and a number of occurrences of the alternate allele on the second strand in the DNA. Note that the reference allele may have a majority allele frequency and the alternate allele has a minority allele frequency.

Moreover, at one or more locations in the DNA where the symmetric normalized odds ratio is greater than the threshold, test results on the tissue sample may not meet one or more desired performance metrics (such as a desired accuracy, confidence, sensitivity and/or specificity). For example, in some embodiments, the confidence metric is the average result for a set of predefined locations in the DNA. Alternatively, at one or more locations in the DNA where the symmetric normalized odds ratio is less than the threshold, test results on the tissue sample may meet one or more desired performance metrics (such as an accuracy, a confidence, a sensitivity and/or a specificity greater than 80%, 85%, 90%, 95% or 98%).

Furthermore, the DNA damage may be associated with formalin fixing and paraffin embedding of the tissue sample. For example, the DNA damage may include: oxidated degradation of guanine to 8-oxoguanine, or formaldehyde-induced DNA and chromatin damage, where the formaldehyde-induced DNA and chromatin damage may include: deamination, depurination, or histone-DNA crosslinks. In some embodiments, the information includes DNA sequences that each correspond to a single strand of DNA from the tissue sample and/or the DNA damage is associated with strand bias.

Computation module 114-1 may use the confidence metric in additional analysis operations. Notably, computation module 114-1 may call variants in the DNA based at least in part on the confidence metric. For example, computation module 114-1 may call variants at one or more locations in the DNA where the symmetric normalized odds ratio is less than the threshold. In some embodiments, the variant calling may use double-strand overlap and/or may use strand-aware rejection of variants. Alternatively or additionally, computation module 114-1 may filter out a subset of the call variants based at least in part on the confidence metric. Notably, computation module 114-1 may filter out call variants at one or more locations in the DNA where the symmetric normalized odds ratio exceeds the threshold. For example, the subset may include false-positive variant calls in the call variants associated with the DNA damage or that are incorrectly labeled as contamination. In some embodiments, the subset may include the variant calls associated with strand bias. Note that the variant calls may include CNVs and/or SNVs.

Alternatively or additionally, computation module 114-1 may output the confidence metric corresponding to one or more locations in the DNA. Then, the one or more of optional control modules 118 may instruct one or more of communication modules 114 (such as communication module 114-1) to provide the confidence metric corresponding to the one or more locations in the DNA to the one or more analysis systems 126. Using the confidence metric corresponding to the one or more locations in the DNA, the one or more analysis systems 126 may adjust one or more sonication parameters that specify subsequent sonication of the tissue sample. In this regard, note that the confidence metric may correspond to a level of DNA fragmentation.

In some embodiments, the analysis techniques may be performed using a look-up table. For example, values of the confidence metric and/or the threshold may be stored in memory module 116-1 as a function of the type of cancer, the number of mutated tumor genetic molecules, the number of tumor genetic molecules and/or the spatial coverage. Alternatively or additionally, the analysis techniques may be performed using a pretrained predictive model, such as a classifier or a regression model. Notably, the information and the threshold may be input to the pretrained predictive model, and the pretrained predictive model may output the confidence metric at or corresponding to one or more locations in the DNA. In general, the pretrained predictive model may include a machine-learning model or a neural network, which was previously trained using a training dataset. Furthermore, the call variants and/or the filtering may be performed using a second pretrained model, such as a second machine-learning model or a second neural network, which was previously trained using a second training dataset. In particular, the information and the confidence metric at or corresponding to one or more locations in the DNA may be input to the second pretrained predictive model, and the second pretrained predictive model may output the call variants or may filter out the subset. In some embodiments, the second pretrained predictive model may use information specifying the sequencing technique (such as a type of DNA probe) and/or a DNA-fragment length as an input. Moreover, generally, one or more features in a given pretrained predictive model may optionally include: a DNA-fragment length, a strand, information associated with a type of DNA damage, an image of a sample, pathology information associated with a sample, histology information associated with a sample, information specifying a dye or staining of a sample, and/or a sample history (such as, in embodiments where a sample is associated with a deceased individual, a time a sample was collected relative to an estimated or known time of death). Note that a given neural network may include or combine: one or more convolutional layers, one or more residual layers and one or more dense or fully connected layers, and where a given node in a given layer in the given neural network may include an activation function, such as: a rectified linear activation function or ReLU, a leaky ReLU, an exponential linear unit or ELU activation function, a parametric ReLU, a tanh activation function, and/or a sigmoid activation function.

After performing at least some of the operations in the analysis techniques, computation module 114-1 may selectively output or provide information specifying or corresponding to the test results on the tissue sample. For example, at one or more locations in the DNA where the confidence metric is less than the threshold (indicating that the tissue sample is not contaminated or degraded and the test results are considered to meet the one or more performance metrics), computation module 114-1 may output test results, e.g., computation module 114-1 may store the test results in memory module 116-1. Note that the test results may include: the confidence metric, mutations or call variants, a cancer classification, such as an indication that the type of cancer is present in the tissue sample (e.g., that a clinical variant has been detected), a treatment recommendation (such as a recommendation for radiation or chemotherapy, a type of chemotherapy, etc.) based at least in part on the indication, and/or another type of test result.

Then, the one or more of optional control modules 118 may instruct one or more of feedback modules 128 (such as feedback module 128-1) to generate a report about an individual associated with the tissue sample (such a computer-aided diagnosis report with feedback, such as the confidence metric, the call variants, the cancer classification, the treatment recommendation, etc.). Furthermore, the one or more of optional control modules 118 may instruct one or more of communication modules 114 (such as communication module 114-1) to return, via network 120 and 122, outputs (such as the computer-aided diagnosis report, etc.) to computer 130 associated with a physician (such as a pathologist) or healthcare provider of the individual.

In these ways, computer system 100 may automatically and accurately assess the confidence of tissue samples associated with the one or more individuals. These capabilities may allow computer system 100 to reliably analyze the DNA in the tissue sample, and/or to detect and diagnose a type of cancer in an automated manner. Moreover, the information determined by computer system 100 (such as the treatment recommendation, e.g., whether or not to perform a surgery, radiation and/or a particular type of chemotherapy) may facilitate or enable improved use of existing treatments (such as precision medicine by selecting a correct medical intervention to treat a type of cancer, e.g., as a companion diagnostic for a prescription drug or a dose of a prescription drug) and/or improved new treatments. Consequently, the analysis techniques may facilitate accurate, value-added use of the measurement or test results, such as genetics analysis of a tissue biopsy sample.

Note that, in some embodiments, computation module 114-1 may determine a quality metric of the tissue sample by aggregating multiple confidence metrics for the molecules in the tissue sample.

While the preceding discussion illustrated the analysis techniques using the symmetric normalized odds ratio, in other embodiments the analysis technique may use another statistical metric to detect the degradation, such as a Fisher’s exact test or a Bayesian statistical technique. Moreover, while preceding discussion illustrated the analysis techniques to selectively detect damage of the DNA associated with or based at least in part on strand bias, more generally the analysis techniques may be used to selectively detect contamination of DNA associated with or based at least in part on stand bias.

We now describe embodiments of the method. FIG. 2 presents a flow diagram illustrating an example of a method 200 for detecting damage of the DNA from a tissue sample, which may be performed by a computer system (such as computer system 100 in FIG. 1). During operation, the computer system may receive information (operation 210) corresponding to identified molecules of the DNA in the tissue sample. For example, the information may include sequence reads. Alternatively or additionally, the information may include Watson and Crick molecules defined using a molecular tag technology, such as the molecular tag technology from Guardant Health of Redwood City, California.

Then, the computer system may determine a symmetric normalized odds ratio (operation 212) based at least in part on the information, where the symmetric normalized odds ratio corresponds to damage of the DNA. Moreover, determining the symmetric normalized odds ratio (operation 212) may include: computing a first odds ratio (operation 214); computing a second odds ratio (operation 216), where a numerator and a denominator in the second odds ratio are reversed relative to the first odds ratio; summing the first odds ratio and the second odds ratio (operation 218); and normalizing the summation (operation 220). Next, the computer system may calculate a confidence metric (operation 222) of one or more of the molecules based at least in part on the symmetric normalized odds ratio and a threshold, where the confidence metric corresponds to a probability that the one or more molecules are identified correctly.

In some embodiments, a given odds ratio in the first odds ratio and the second odds ratio may be computed based at least in part on: a number of occurrences of a reference allele on a first strand in the DNA; a number of occurrences of the reference allele on a second strand in the DNA; a number of occurrences of a alternate allele on the first strand in the DNA; and a number of occurrences of the alternate allele on the second strand in the DNA. Note that the reference allele may have a majority allele frequency and the alternate allele has a minority allele frequency.

Note that the DNA damage may be associated with formalin fixing and paraffin embedding of the tissue sample. For example, the DNA damage may include: oxidated degradation of guanine to 8-oxoguanine, or formaldehyde-induced DNA and chromatin damage, where the formaldehyde-induced DNA and chromatin damage may include: deamination, depurination, or histone-DNA crosslinks. In some embodiments, the information includes DNA sequences that each correspond to a single strand of DNA from the tissue sample and/or the DNA damage is associated with strand bias.

In some embodiments, the computer system may optional perform one or more additional operations (operation 224). For example, the computer system may call variants in the DNA based at least in part on the confidence metric. Furthermore, the computer system may filter out a subset of the call variants based at least in part on the confidence metric. For example, the subset may include false-positive variant calls in the call variants associated with the DNA damage or that are incorrectly labeled as contamination. Alternatively or additionally, the subset may include the variant calls associated with strand bias. Note that the variant calls may include CNVs and/or SNVs.

Additionally, the computer system may adjust one or more sonication parameters for subsequent sonication of the tissue sample based at least in part on the confidence metric. Note that the confidence metric may correspond to a level of DNA fragmentation.

In some embodiments, the computer system may determine a quality metric of the tissue sample by aggregating multiple confidence metrics for the molecules in the tissue sample.

In some embodiments of method 200, there may be additional or fewer operations. Furthermore, the order of the operations may be changed, and/or two or more operations may be combined into a single operation.

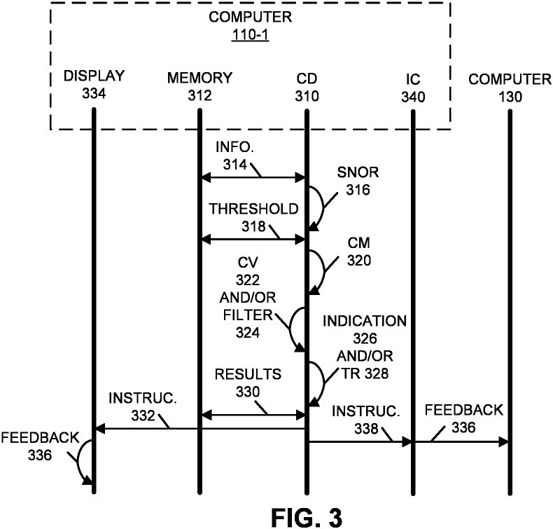

Embodiments of the analysis techniques are further illustrated in FIG. 3, which presents a drawing illustrating an example of communication among components in computer system 100. In FIG. 3, a computation device (CD) 310 (such as a processor or a GPU) in computer 110-1 may access, in memory 312 in computer 110-1, information 314 corresponding to a sample that is associated with a tissue biopsy. For example, information 314 may be the result of sequencing of the DNA from a tissue sample and molecular annotation that collapses sequencing reads into molecules. Thus, information 314 may corresponding to molecules of the DNA in the tissue sample.

After receiving information 314, computation device 310 may determine a symmetric normalized odds ratio (SNOR) 316 based at least in part on information 314. Moreover, determining the symmetric normalized odds ratio 316 may include: computing a first odds ratio; computing a second odds ratio, where a numerator and a denominator in the second odds ratio are reversed relative to the first odds ratio; summing the first odds ratio and the second odds ratio; and normalizing the summation. Next, computation device 310 may calculate a confidence metric (CM) 320 of one or more of the molecules based at least in part on the symmetric normalized odds ratio 316 and a threshold 318, where the confidence metric corresponds to a probability that the one or more molecules are identified correctly, and where the symmetric normalized odds ratio 316 and/or a threshold 318 may be access in memory 312.

Moreover, based at least in part on the confidence metric 320, computation device 310 may call variants (CV) 322 in the DNA and/or may filter 324 the call variants 322. Alternatively or additionally, computation device 310 may determine an indication 326 that a type of cancer is present in the tissue sample and/or a treatment recommendation (TR) 328 based at least in part on the indication 326.

After or while performing the preceding operations, computation device 310 may store results 330, including the confidence metric 320, the call variants 322, the filtered call variants, indication 326 and/or treatment recommendation 328, in memory 312. Next, computation device 310 may provide instructions 332 to a display 334 in computer 110-1 to display feedback 336, such as results 330 (and, more generally, a computer-aided diagnosis report). Alternatively or additionally, computation device 310 may provide instructions 338 to an interface circuit 340 in computer 110-1 to provide feedback 336 to another computer or electronic device, such as computer 130.

While FIG. 3 illustrates communication between components using unidirectional or bidirectional communication with lines having single arrows or double arrows, in general the communication in a given operation in this figure may involve unidirectional or bidirectional communication.

We now further describe embodiments of the analysis techniques. Variant calling may be difficult in archival tissue samples, such as those that have been formalin-fixed and paraffin-embedded. This is because formalin-fixed and paraffin-embedding and long-term storage often introduce a variety of chemical changes to DNA that can be detected as mutations during sequencing. Therefore, it is useful to distinguish between real mutations and DNA damage that results from formalin-fixed and paraffin-embedded storage. Several types of formalin-fixed and paraffin-embedded-related DNA damage affect only one strand of the DNA, which means that analysis technique that identifies mutations that are heavily overrepresented on one DNA strand (or strand-biased) may be used to distinguish between true mutations in tissue samples (such as tumor samples) and DNA-damage-related mutations.

The disclosed analysis techniques may be used to detect strand bias (e.g., for SNVs) that is associated with DNA damage. Notably, the analysis techniques may be based at least in part on a symmetric normalized odds ratio and may facilitate the identification of SNVs caused by certain types of DNA damage, such as DNA damage associated with formalin-fixed and paraffin-embedding preservation and storage of tissue samples. The resulting confidence metric may be used to filter ‘false positive’ variants caused by DNA damage (such as filtering false positive germline contamination signals) rather than true mutations. In some embodiments, the symmetric normalized odds ratio is calculated using Watson and Crick molecules, which may identify variants that were significantly biased in the input tissue sample before PCR and/or sequencing.

For example, the analysis techniques may be used to identify strand-biased variants associated with false-positive germline contamination (e.g., from variants that are incorrectly identified as being associated with another tissue sample because of damage associated with formalin-fixed and paraffin-embedding preservation and storage). Germline contamination may be calculated as the number of known common germline variants that occur at lower allele frequencies (MAFs) than expected for germline variants (such as annotated common germline variants having MAFs less than 15% and with contaminated variants occurring in at least six genes, as opposed to typically germline variants that have allele frequencies of 50-100%). These low-MAF germline variants may represent or may be associated with the introduction of a small amount of another tissue sample. However, large amounts of DNA damage can also generate variants with the same phenotype (low-MAF variants annotated as common germlines), causing false-positive contamination flags. For example, a high tumor mutation burden, Aneuploidy and/or formalin-fixed and paraffin-embedding preservation and storage can result in specific types of DNA damage that appear to be variants. Therefore, a strand bias filter based at least in part in the confidence metric in the analysis techniques may reduce or eliminate false-positive contaminating variants, and may rescue some tissue samples that were erroneously labeled as contaminated.

In some embodiments, the confidence metric may facilitate a variety of adaptive operational and/or bioinformatic processing, such as: calling variants, filtering variants, and/or adjusting subsequent sonication of the tissue sample. In general, DNA damage is associated with a variety of challenges in variant calling and sample processing, and the confidence metric may provide a way to assess DNA damage holistically, which may provide significant performance benefits in traditionally challenging sequencing samples. Moreover, because the majority of clinical cancer samples are stored in an archival format, the confidence metric and processes informed by it may provide significant value in terms of the use of available tissue-sample volume, as well as an ability to perform high-quality sequencing and analysis of lower-quality tissue samples.

In the discussion that follows, the specific damage associated with formalin-fixed and paraffin-embedding preservation and storage of tissue samples is used as an illustrative example of the degradation that can be detected using the analysis techniques. As discussed previously, oxidative degradation of guanine to 8-oxoguanine is a common preservation and storage-related artifact. Unlike guanine, oxidated degradation of guanine to 8-oxoguanine may preferentially bind to adenine rather than cytosine. This may result in guanine to thymine and cytosine to adenine transitions in sequencing data. These lesions are also typically heavily strand-biased, with a given oxidated degradation of guanine to 8-oxoguanine-associated variant occurring on only one strand. The disclosed analysis techniques may be used to identify and/or filter strand-biased contaminating variants, thereby reducing human review rates by reducing or eliminating fixed and paraffin-embedding-related false-positive contamination calls.

Existing strand-bias calculation are often based on read-sequence ratios, which are not well correlated with molecular values. The disclosed symmetric normalized odds ratio may calculate the relative odds of a variant being strand-biased, and may be based at least in part on molecules. Using the contingency table counts shown in Table 1, the first odds ratio (OR) may be calculated as

$$O R = \frac{R W ⋅ A C}{R C ⋅ A W} ,$$

the second or inverse odds ratio (OR⁻¹) may be calculated as

$$O {R}^{- 1} = \frac{R C ⋅ A W}{R W ⋅ A C} ,$$

a reference ratio (refRatio) may be calculated as

$$r e f R a t i o = \frac{min \left(R W , R C\right)}{max \left(R W , R C\right)} ,$$

and alternate ratio (altRatio) may be calculated as

$$a l t R a t i o = \frac{min \left(A W , A C\right)}{max \left(A W , A C\right)} .$$

Then, the symmetric normalized odds ratio (SNOR) may be determined as SNOR = In(OR + OR⁻¹) + In(refRatio) - In(altRatio).

Note that the symmetric normalized odds ratio may be used for variant alleles (or a non-reference base) in the DNA.

TABLE 1

| | | |
|---|---|---|
| | Watson | Crick |
| Reference Allele | RW | RC |
| Alternate Allele | AW | AC |

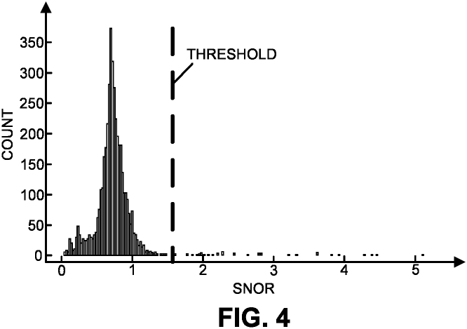

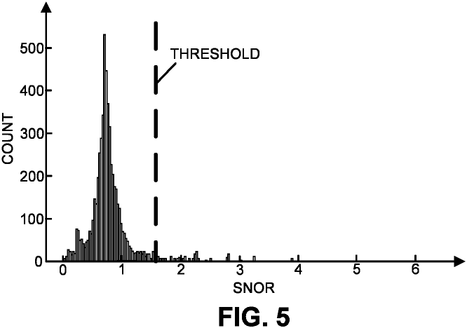

Strand-bias filtering of false-positive contamination flags using the confidence metric is illustrated in FIGS. 4 and 5, which present drawings illustrating examples of the symmetric normalized odds ratio and the threshold for tissue samples. Notably, in FIGS. 4 and 5, the symmetric normalized odds ratio is shown for, respectively, 4,176 and 6,500 randomly sampled SNVs in normal tissue. Note that the distribution of values is roughly normal with a long tail to the right indicating highly strand-biased variants. The dashed vertical lines show the threshold at the mean plus three standard deviations or 1.57. Thus, for normal tissue samples, most variants have values of the symmetric normalized odds ratio that are less than 1.57, and the long tail of values likely represent variants caused by fixed and paraffin-embedding preservation and storage of tissue samples and/or other technical effects rather than true variants.

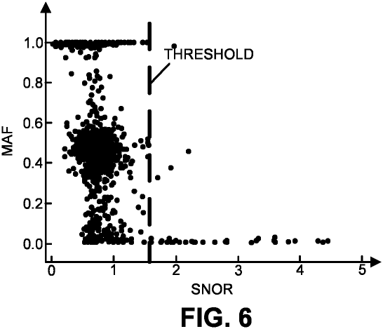

FIG. 6 presents a drawing illustrating an example of the MAF, the symmetric normalized odds ratio and the threshold (1.57) for tissue samples. Notably, in FIG. 6, the MAF as a function of the symmetric normalized odds ratio is shown for 4,176 randomly sampled SNVs. The dashed vertical line shows the threshold at the mean plus three standard deviations or 1.57. The results shown that nearly all strand-biased variants occur at low MAFs, as expected from damage-induced variants. Among the assessed SNVs (not all of which are shown in FIG. 6), there are 81 strand-biased variants, with 12 (14.8%) oxidated degradation of guanine to 8-oxoguanine-related variants and 34 (41.98%) formalin-fixed and paraffin-embedding-related variants. The overall prevalence is 6.2% for oxidated degradation of guanine to 8-oxoguanine and 38% formalin-fixed and paraffin-embedding-related. Of the strand-biased variants, 11 were flagged as contaminated, two of which are oxidated degradation of guanine to 8-oxoguanine. There were 81 total contamination flags, so strand bias represents 13.6%. Note that none strand-biased variants are defined as call equal to 1.

Thus, the symmetric normalized odds ratio cutoff at 1.57 is enriched for low-MAF variants associated with oxidated degradation of guanine to 8-oxoguanine. This includes oxidated degradation of guanine to 8-oxoguanine-related variants. (It is currently unclear what drives other strand-biased variants.) None of the examined strand-biased variants were call equal to 1. Consequently, a threshold of 1.57 (three standard deviations from the mean) filters out low-MAF contaminated variants that are likely caused by DNA damage.

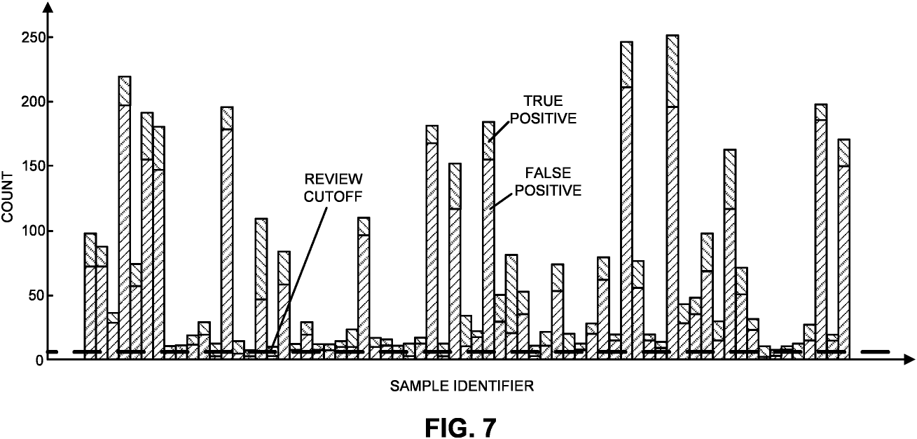

FIG. 7 presents a drawing illustrating an example of an impact of a confidence metric on reviews of tissue samples. Notably, FIG. 7 shows false-positive and true-positive contaminated gene counts associated with strand bias. The dashed horizontal line is the review cutoff. False positives below the review cutoff would be recovered. For the clinical samples with germline contamination reviews, a symmetric normalized odds ratio filter of 1.57 eliminates 11/67 reviews (16.4%). Therefore, stand-bias cutoff or filtering reduces review rates.

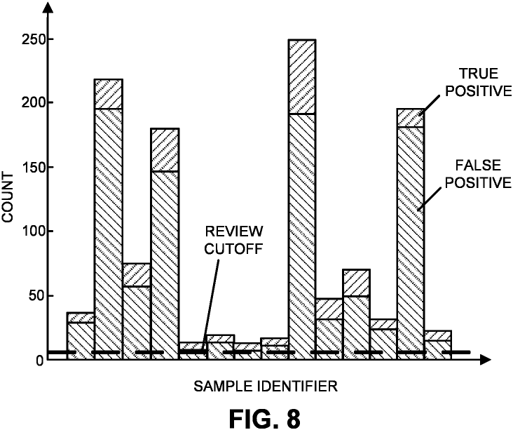

Moreover, as shown in FIG. 8, which presents a drawing illustrating an example of an impact of a confidence metric on reviews of tissue samples, the use of stand-bias cutoff or filtering does not result in false-negative reviews (such as the elimination of verified contamination events). Notably, FIG. 8 shows false-positive and true-positive contaminated gene counts associated with strand bias for 14 clinical samples with contaminations verified as having known within-batch donors. The dashed horizontal line is the review cutoff. False positives below the review cutoff would be recovered. The strand-bias filter retains all 14 true-positive contamination reviews and does not result in any false-negative germline contamination flags. Thus, the calculations for germline contaminations may omit variants with symmetric normalized odds ratios greater than 1.57.

In summary, formalin-fixed and paraffin-embedding damage often results in false-positive contamination reviews and contributes to review rates. Moreover, oxidated degradation of guanine to 8-oxoguanine variants tend to be strand-biased. The with symmetric normalized odds ratio effectively identifies strand-biased variants, which appear to be primarily caused by formalin-fixed and paraffin-embedding-related damage. Furthermore, filtering contaminating variants with symmetric normalized odds ratios greater than 1.57 may reduce contamination review rates, e.g., by 16%. The risk of germline contamination false negatives caused by this filtering is low. Thus, a symmetric normalized odds ratio-based filter may be used to remove contamination-related variants, thereby preventing these variants from being counted, e.g., in germline contamination calculations.

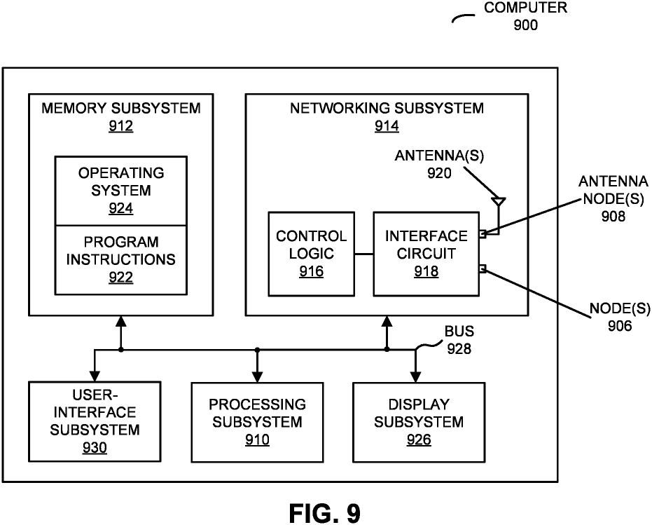

FIG. 9 presents a block diagram illustrating an example of a computer 900, e.g., in a computer system (such as computer system 100 in FIG. 1), in accordance with some embodiments. Computer 900 may regulate various aspects sample preparation, sequencing, and/or analysis, such as: determining the dynamic confidence metric, comparing the dynamic confidence metric to a threshold, and selectively providing an indication that a type of cancer is present in a sample. In some examples, computer 401 is configured to perform sample preparation and sample analysis, including nucleic acid sequencing.

Computer 900 may include: one of computers 110. This computer may include processing subsystem 910, memory subsystem 912, and networking subsystem 914. Processing subsystem 910 includes one or more devices configured to perform computational operations. For example, processing subsystem 910 can include one or more microprocessors (such as a single-core or a multi-core processor), ASICs, microcontrollers, programmable-logic devices, GPUs and/or one or more DSPs. Processing subsystem 910 may perform parallel processing of one or more operations in the analysis techniques. Note that a given component in processing subsystem 910 are sometimes referred to as a ‘computation device’.

Memory subsystem 912 includes one or more devices for storing data and/or instructions for processing subsystem 910 and networking subsystem 914. For example, memory subsystem 912 can include dynamic random access memory (DRAM), static random access memory (SRAM), flash and/or other types of memory. In some embodiments, instructions for processing subsystem 910 in memory subsystem 912 include: program instructions or sets of instructions (such as program instructions 922 or operating system 924), which may be executed by processing subsystem 910. Note that the one or more computer programs or program instructions may constitute a computer-program mechanism. Moreover, instructions in the various program instructions in memory subsystem 912 may be implemented in: a high-level procedural language, an object-oriented programming language, and/or in an assembly or machine language. Furthermore, the programming language may be compiled or interpreted, e.g., configurable or configured (which may be used interchangeably in this discussion), to be executed by processing subsystem 910. Thus, program instructions 922 may be precompiled for use with computer 900 or may be compiled at runtime. In some embodiments, program instructions 922 are stored or embodied on a type of non-transitory machine-readable medium, which may include a portable non-transitory machine-readable medium (e.g., a floppy disk, a flexible disk, hard disk, magnetic tape, any other magnetic medium, a CD-ROM, DVD or DVD-ROM, any other optical medium, punch cards, paper tape, any other physical storage medium with patterns of holes, a RAM, a ROM, a PROM and EPROM, a FLASH-EPROM, any other memory chip or cartridge, or any other medium from which a computer may read programming code and/or data).

In addition, memory subsystem 912 can include mechanisms for controlling access to the memory. In some embodiments, memory subsystem 912 includes a memory hierarchy that includes one or more caches coupled to a memory in computer 900. In some of these embodiments, one or more of the caches is located in processing subsystem 910.

In some embodiments, memory subsystem 912 is coupled to one or more high-capacity mass-storage devices (not shown), which may be external to computer 900 and/or remotely located (and, thus, accessed via a network). For example, memory subsystem 912 can be coupled to a magnetic or optical drive, a solid-state drive, or another type of mass-storage device. In these embodiments, memory subsystem 912 can be used by computer 900 as fast-access storage for often-used data, while the mass-storage device is used to store less frequently used data. Note that data may be transferred from one location to another using, e.g., a network (such as the Internet and/or an intra-net) or physical data transfer (e.g., using a hard drive, thumb drive, or other data-storage device).

Networking subsystem 914 includes one or more devices configured to couple to and communicate on a wired and/or wireless network (i.e., to perform network operations), including: control logic 916, an interface circuit 918 and one or more antennas 920 (or antenna elements). (While FIG. 9 includes one or more antennas 920, in some embodiments computer 900 includes one or more nodes, such as antenna nodes 908, e.g., a metal pad or a connector, which can be coupled to the one or more antennas 920, or nodes 906, which can be coupled to a wired or optical connection or link. Thus, computer 900 may or may not include the one or more antennas 920. Note that the one or more nodes 906 and/or antenna nodes 908 may constitute input(s) to and/or output(s) from computer 900.) For example, networking subsystem 914 can include a Bluetooth™ networking system, a cellular networking system (e.g., a 3G/4G/5G network such as UMTS, LTE, etc.), a universal serial bus (USB) networking system, a networking system based on the standards described in IEEE 802.11 (e.g., a Wi-Fi® networking system), an Ethernet networking system, and/or another networking system.

Networking subsystem 914 includes processors, controllers, radios/antennas, sockets/plugs, and/or other devices used for coupling to, communicating on, and handling data and events for each supported networking system. Note that mechanisms used for coupling to, communicating on, and handling data and events on the network for each network system are sometimes collectively referred to as a ‘network interface’ for the network system. Moreover, in some embodiments a ‘network’ or a ‘connection’ between the electronic devices does not yet exist. Therefore, computer 900 may use the mechanisms in networking subsystem 914 for performing simple wireless communication between electronic devices, e.g., transmitting advertising or beacon frames and/or scanning for advertising frames transmitted by other electronic devices.

Within computer 900, processing subsystem 910, memory subsystem 912, and networking subsystem 914 are coupled together using bus 928. Bus 928 may include an electrical, optical, and/or electro-optical connection that the subsystems can use to communicate commands and data among one another. Although only one bus 928 is shown for clarity, different embodiments can include a different number or configuration of electrical, optical, and/or electro-optical connections among the subsystems.

In some embodiments, computer 900 includes a display subsystem 926 for displaying information on a display, which may include a display driver and the display, such as a liquid-crystal display, a multi-touch touchscreen, etc. Moreover, computer 900 may include a user-interface subsystem 930, such as: a mouse, a keyboard, a trackpad, a stylus, a voice-recognition interface, and/or another human-machine interface. Note that user-interface subsystem 930 may include graphical user interface (GUI) and/or a web-based user interface

Additional details relating to computer systems and networks, data structures, databases, and computer program products are also provided in, for example, Peterson, Computer Networks: A Systems Approach, Morgan Kaufmann, 5th Ed. (2011), Kurose, Computer Networking: A Top-Down Approach, Pearson, 7^{th} Ed. (2016), Elmasri, Fundamentals of Database Systems, Addison Wesley, 6th Ed. (2010), Coronel, Database Systems: Design, Implementation, & Management, Cengage Learning, 11^{th} Ed. (2014), Tucker, Programming Languages, McGraw-Hill Science/Engineering/Math, 2nd Ed. (2006), and Rhoton, Cloud Computing Architected: Solution Design Handbook, Recursive Press (2011), each of which is hereby incorporated by reference in its entirety.

Computer 900 can be (or can be included in) any electronic device with at least one network interface. For example, computer 900 can be (or can be included in): a desktop computer, a laptop computer, a subnotebook/netbook, a server, a supercomputer, a tablet computer, a smartphone, a cellular telephone, a consumer-electronic device, a portable computing device, communication equipment, and/or another electronic device.

Although specific components are used to describe computer 900, in alternative embodiments, different components and/or subsystems may be present in computer 900. For example, computer 900 may include one or more additional processing subsystems, memory subsystems, networking subsystems, and/or display subsystems. Additionally, one or more of the subsystems may not be present in computer 900. Moreover, in some embodiments, computer 900 may include one or more additional subsystems that are not shown in FIG. 9. Also, although separate subsystems are shown in FIG. 9, in some embodiments some or all of a given subsystem or component can be integrated into one or more of the other subsystems or component(s) in computer 900. For example, in some embodiments program instructions 922 are included in operating system 924 and/or control logic 916 is included in interface circuit 918.

Moreover, the circuits and components in computer 900 may be implemented using any combination of analog and/or digital circuitry, including: bipolar, PMOS and/or NMOS gates or transistors. Furthermore, signals in these embodiments may include digital signals that have approximately discrete values and/or analog signals that have continuous values. Additionally, components and circuits may be single-ended or differential, and power supplies may be unipolar or bipolar.

An integrated circuit may implement some or all of the functionality of networking subsystem 914 and/or computer 900. The integrated circuit may include hardware and/or software mechanisms that are used for transmitting signals from computer 900 and receiving signals at computer 900 from other electronic devices. Aside from the mechanisms herein described, radios are generally known in the art and hence are not described in detail. In general, networking subsystem 914 and/or the integrated circuit may include one or more radios.

In some embodiments, an output of a process for designing the integrated circuit, or a portion of the integrated circuit, which includes one or more of the circuits described herein may be a computer-readable medium such as, e.g., a magnetic tape or an optical or magnetic disk or solid state disk. The computer-readable medium may be encoded with data structures or other information describing circuitry that may be physically instantiated as the integrated circuit or the portion of the integrated circuit. Although various formats may be used for such encoding, these data structures are commonly written in: Caltech Intermediate Format (CIF), Calma GDS II Stream Format (GDSII), Electronic Design Interchange Format (EDIF), OpenAccess (OA), or Open Artwork System Interchange Standard (OASIS). Those of skill in the art of integrated circuit design can develop such data structures from schematics of the type detailed above and the corresponding descriptions and encode the data structures on the computer-readable medium. Those of skill in the art of integrated circuit fabrication can use such encoded data to fabricate integrated circuits that include one or more of the circuits described herein.

While some of the operations in the preceding embodiments were implemented in hardware or software, in general the operations in the preceding embodiments can be implemented in a wide variety of configurations and architectures. Therefore, some or all of the operations in the preceding embodiments may be performed in hardware, in software or both. For example, at least some of the operations in the analysis techniques may be implemented using program instructions 922, operating system 924 (such as a driver for interface circuit 918) or in firmware in interface circuit 918. Thus, the analysis techniques may be implemented at runtime of program instructions 922. Alternatively or additionally, at least some of the operations in the analysis techniques may be implemented in a physical layer, such as hardware in interface circuit 918.

In some embodiments, the confidence metric may be used to detect RNA contamination in DNA. Notably, because RNA and DNA may be processed or prepared on the same machine(s) or in similar workflows, there may be cross-contamination between the two analytes. Because the RNA preparations are single-stranded, contaminating RNA into the DNA workflow may be represented by an introduction of bias in one strand, which is what symmetric normalized odds ratio detects.

In some embodiments, the introduction of single-stranded DNA into the DNA workflow may be represented by an introduction of bias in one strand, which is what symmetric normalized odds ratio detects. In some embodiments, the confidence metric may be used to detect recovery of single-stranded DNA from enzymatic or chemical treatment, such as with bisulfite treatment, the use of the APOBEC family enzymes that deaminate cytosine bases to uracil in single-stranded DNA, or a fragmentation method. These methods along with the confidence metric may be used as a tool in methylation analysis. In some embodiments, the confidence metric may be used to detect molecular recovery and/or topology in a hybrid workflow comprising the preparation and analysis of single-stranded DNA and double-stranded DNA.

Moreover, in some embodiments, the analysis techniques allow a given read budget during analysis or sequencing to achieve improved variant calls or identification. Notably, the number of reads needed to correctly identify or call a variant may be reduced. This capability may allow the given read budget to provide improved results (which is sometimes referred to as ‘performance’), which may make an analysis product more affordable for a given performance. In particular, the analysis techniques may use one or more odds-ratio filters to filter out or remove one or more variants that are associated with DNA damage, thereby reducing the number of reads that are needed to correctly identify or call the remaining variants. Therefore, the analysis techniques may allow the given read budget to be reallocated to address other issues in the analysis, such as issues that affect the accuracy of somatic, epigenomic and/or whole exome variant calling in a tissue sample. Thus, the analysis techniques may allow the given read budget to be used or leveraged for improved performance.

Factors of a read budget can include read depth, panel size, and/or limit of detection. For example, a read budget of 3,000,000,000 reads can be allocated as 150,000 bases at an average read depth of 20,000 reads/base. Read depth can refer to number of molecules producing a read at a locus. In the present disclosure, the reads at each base can be allocated between bases in the backbone region of the panel, at a first average read depth and bases in the hotspot region of the panel, at a deeper read depth. In some embodiments, a sample is sequenced to a read depth determined by the amount of nucleic acid present in a sample. In some embodiments, a sample is sequenced to a set read depth, such that samples comprising different amounts of nucleic acid are sequenced to the same read depth. For example, a sample comprising 300 ng of nucleic acids can be sequenced to a read depth ⅒ that of a sample comprising 30 ng of nucleic acids. In some embodiments, nucleic acids from two or more different subjects can be added together at a ratio based on the amount of nucleic acids obtained from each of the subjects.

By way of non-limiting example, if a read budget consists of 100,000 read counts for a given sample, those 100,000 read counts will be divided between reads of backbone regions and reads of hotspot regions. Allocating a large number of those reads (e.g., 90,000 reads) to backbone regions will result in a small number of reads (e.g., the remaining 10,000 reads) being allocated to hotspot regions. Conversely, allocating a large number of reads (e.g., 90,000 reads) to hotspot regions will result in a small number of reads (e.g., the remaining 10,000 reads) being allocated to backbone regions. Thus, a skilled worker can allocate a read budget to provide desired levels of sensitivity and specificity. In certain embodiments, the read budget can be between 100,000,000 reads and 100,000,000,000 reads, e.g., between 500,000,000 reads and 50,000,000,000 reads, or between about 1,000,000,000 reads and 5,000,000,000 reads across, for example, 20,000 bases to 100,000 bases.

As another example, a read budget may include 90 million (M) sequence clusters per sample, 55 M of which may be allocated for DNA genomic analysis, 10 M for epigenomic analysis, 20 M for whole exome analysis, and 5 M for RNA analysis. Such samples can then be multiplexed with additional samples. Filtering for strand bias can decrease this budget by at least 1%, 2%, 3%, 4%, 5%, 10%, 15%, or more. In some embodiments, the read budget is decreased from 1%-5%. In some embodiments, the read budget is decreased from 2%-4%. In some embodiments, the read budget is decreased from 3%-6%. In some embodiments, the read budget is decreased from 5%-10%. Decreasing the read budget for one panel may allow for more read budget to be reallocated to another panel.

In some embodiments, the method provides denoised data going into the variant calling algorithm. The less noise in the input, the more confident one can be in analyzing “borderline molecules.” For example, instead of having a higher threshold for confidence in oxoG related variants to account for DNA damage, one can exclude it and have a similar variant calling threshold as other non-DNA-damage-related variant classes.

### Samples

While tissue biopsy is used as illustrations of a sample in the present disclosure, more generally a sample can be any biological sample isolated from a subject. Samples can include body tissues, whole blood, platelets, serum, plasma, stool, red blood cells, white blood cells or leucocytes, endothelial cells, tissue biopsies (e.g., biopsies from known or suspected solid tumors), cerebrospinal fluid, synovial fluid, lymphatic fluid, ascites fluid, interstitial or extracellular fluid (e.g., fluid from intercellular spaces), gingival fluid, crevicular fluid, bone marrow, pleural effusions, cerebrospinal fluid, saliva, mucous, sputum, semen, sweat, and/or urine. Samples are preferably body fluids, particularly blood and fractions thereof, and urine. Such samples include nucleic acids shed from tumors. The nucleic acids can include DNA and RNA and can be in double and single-stranded forms. A sample can be in the form originally isolated from a subject or can have been subjected to further processing to remove or add components, such as cells, enrich for one component relative to another, or convert one form of nucleic acid to another, such as RNA to DNA or single-stranded nucleic acids to double-stranded. Thus, e.g., a body fluid for analysis is plasma or serum containing cell-free nucleic acids, e.g., cell-free DNA. In some embodiments, the analysis techniques include obtaining the sample from a subject. Essentially any sample type is optionally utilized. In certain embodiments, e.g., the sample is tissue, blood, plasma, serum, sputum, urine, semen, vaginal fluid, feces, synovial fluid, spinal fluid, saliva, and/or the like. Typically, the subject is a mammalian subject (e.g., a human subject). In some embodiments, the sample is blood. In some embodiments, the sample is plasma. In some embodiments, the sample is serum.

In some embodiments, the sample volume of body fluid taken from a subject depends on the desired read depth for sequenced regions. Exemplary volumes are about 0.4-40 ml, about 5-20 ml, about 10-20 ml. For example, the volume can be about 0.5 ml, about 1 ml, about 5 ml, about 10 ml, about 20 ml, about 30 ml, about 40 ml, or more milliliters. A volume of sampled plasma is typically between about 5 ml to about 20 ml.

The sample can include various amounts of nucleic acid. Typically, the amount of nucleic acid in a given sample is equates with multiple genome equivalents. For example, a sample of about 30 ng DNA can contain about 10,000 (10⁴) haploid human genome equivalents and, in the case of cfDNA, about 200 billion (2×10¹¹) individual polynucleotide molecules. Similarly, a sample of about 100 ng of DNA can contain about 30,000 haploid human genome equivalents and, in the case of cfDNA, about 600 billion individual molecules.

In some embodiments, a sample includes nucleic acids from different sources, e.g., from cells and from cell-free sources (e.g., blood samples, etc.). Typically, a sample includes nucleic acids carrying mutations. For example, a sample optionally includes DNA carrying germline mutations and/or somatic mutations. Alternatively or additionally, a sample includes DNA carrying cancer-associated mutations (e.g., cancer-associated somatic mutations). In some embodiments, the sample includes cell-free DNA (i.e., cfDNA sample). In some embodiments, the cfDNA sample includes circulating tumor nucleic acids.

Exemplary amounts of cell-free nucleic acids in a sample before amplification typically range from about 1 femtogram (fg) to about 1 microgram (µg), e.g., about 1 picogram (pg) to about 200 nanogram (ng), about 1 ng to about 100 ng, or about 10 ng to about 1000 ng. In some embodiments, a sample includes up to about 600 ng, up to about 500 ng, up to about 400 ng, up to about 300 ng, up to about 200 ng, up to about 100 ng, up to about 50 ng, or up to about 20 ng of cell-free nucleic acid molecules. Optionally, the amount is at least about 1 fg, at least about 10 fg, at least about 100 fg, at least about 1 pg, at least about 10 pg, at least about 100 pg, at least about 1 ng, at least about 10 ng, at least about 100 ng, at least about 150 ng, or at least about 200 ng of cell-free nucleic acid molecules. In certain embodiments, the amount is up to about 1 fg, about 10 fg, about 100 fg, about 1 pg, about 10 pg, about 100 pg, about 1 ng, about 10 ng, about 100 ng, about 150 ng, or about 200 ng of cell-free nucleic acid molecules. In some embodiments, the analysis techniques include obtaining between about 1 fg to about 200 ng cell-free nucleic acid molecules from samples. In certain embodiments, the analysis techniques include obtaining between about 5 ng to about 30 ng of cell-free nucleic acid molecules from samples. In certain embodiments, the analysis techniques include obtaining between about 5 ng to about 100 ng of cell-free nucleic acid molecules from samples. In certain embodiments, the analysis techniques include obtaining between about 5 ng to about 150 ng of cell-free nucleic acid molecules from samples. In certain embodiments, the analysis techniques include obtaining between about 5 ng to about 200 ng of cell-free nucleic acid molecules from samples. In some embodiments, the amount is up to about 100 ng of cell-free nucleic acid molecules from samples. In some embodiments, the amount is up to about 150 ng of cell-free nucleic acid molecules from samples. In some embodiments, the amount is up to about 200 ng of cell-free nucleic acid molecules from samples. In some embodiments, the amount is up to about 250 ng of cell-free nucleic acid molecules from samples. In some embodiments, the amount is up to about 300 ng of cell-free nucleic acid molecules from samples. In some embodiments, the analysis techniques include obtaining between about 1 fg to about 200 ng cell-free nucleic acid molecules from samples.

Cell-free nucleic acids typically have a size distribution of between about 100 nucleotides in length and about 500 nucleotides in length, with molecules of about 110 nucleotides in length to about 230 nucleotides in length representing about 90% of molecules in the sample, with a mode of about 168 nucleotides in length and a second minor peak in a range between about 240 to about 440 nucleotides in length. In certain embodiments, cell-free nucleic acids are from about 160 to about 180 nucleotides in length, or from about 320 to about 360 nucleotides in length, or from about 440 to about 480 nucleotides in length.

In some embodiments, cell-free nucleic acids are isolated from bodily fluids through a partitioning operation in which cell-free nucleic acids, as found in solution, are separated from intact cells and other non-soluble components of the bodily fluid. In some of these embodiments, partitioning includes analysis techniques such as centrifugation or filtration. Alternatively, cells in bodily fluids are lysed, and cell-free and cellular nucleic acids processed together. Generally, after addition of buffers and wash operations, cell-free nucleic acids are precipitated with, e.g., an alcohol. In certain embodiments, additional clean-up operations are used, such as silica-based columns to remove contaminants or salts. Non-specific bulk carrier nucleic acids, e.g., are optionally added throughout the reaction to optimize certain aspects of the exemplary procedure, such as yield. After such processing, samples typically include various forms of nucleic acids including double-stranded DNA, single-stranded DNA and/or single-stranded RNA. Optionally, single stranded DNA and/or single stranded RNA are converted to double stranded forms so that they are included in subsequent processing and analysis operations.

### Nucleic Acid Tags

In some embodiments, the nucleic acid molecules (from the sample of polynucleotides) may be tagged with sample indexes and/or molecular barcodes (referred to generally as ‘tags’). Tags may be incorporated into or otherwise joined to adapters by chemical synthesis, ligation (e.g., blunt-end ligation or sticky-end ligation), or overlap extension PCR, among other methods. Such adapters may be ultimately joined to the target nucleic acid molecule. In other embodiments, one or more rounds of amplification cycles (e.g., PCR amplification) are generally applied to introduce sample indexes to a nucleic acid molecule using conventional nucleic acid amplification methods. The amplifications may be conducted in one or more reaction mixtures (e.g., a plurality of microwells in an array). Molecular barcodes and/or sample indexes may be introduced simultaneously, or in any sequential order. In some embodiments, molecular barcodes and/or sample indexes are introduced prior to and/or after sequence capturing operations are performed. In some embodiments, only the molecular barcodes are introduced prior to probe capturing and the sample indexes are introduced after sequence capturing operations are performed. In some embodiments, both the molecular barcodes and the sample indexes are introduced prior to performing probe-based capturing operations. In some embodiments, the sample indexes are introduced after sequence capturing operations are performed. In some embodiments, molecular barcodes are incorporated to the nucleic acid molecules (e.g., cfDNA molecules) in a sample through adapters via ligation (e.g., blunt-end ligation or sticky-end ligation). In some embodiments, sample indexes are incorporated to the nucleic acid molecules (e.g., cfDNA molecules) in a sample through overlap extension PCR. Typically, sequence capturing protocols involve introducing a single-stranded nucleic acid molecule complementary to a targeted nucleic acid sequence, e.g., a coding sequence of a genomic region and mutation of such region is associated with a cancer type.

In some embodiments, the tags may be located at one end or at both ends of the sample nucleic acid molecule. In some embodiments, tags are predetermined or random or semi-random sequence oligonucleotides. In some embodiments, the tags may be less than about 500, 200, 100, 50, 20, 10, 9, 8, 7, 6, 5, 4, 3, 2, or 1 nucleotides in length. The tags may be linked to sample nucleic acids randomly or non-randomly.

In some embodiments, each sample is uniquely tagged with a sample index or a combination of sample indexes. In some embodiments, each nucleic acid molecule of a sample or sub-sample is uniquely tagged with a molecular barcode or a combination of molecular barcodes. In other embodiments, a plurality of molecular barcodes may be used such that molecular barcodes are not necessarily unique to one another in the plurality (e.g., non-unique molecular barcodes). In these embodiments, molecular barcodes are generally attached (e.g., by ligation) to individual molecules such that the combination of the molecular barcode and the sequence it may be attached to creates a unique sequence that may be individually tracked. Detection of non-uniquely tagged molecular barcodes in combination with endogenous sequence information (e.g., the beginning (start) and/or end (stop) portions corresponding to the sequence of the original nucleic acid molecule in the sample, sub-sequences of sequence reads at one or both ends, length of sequence reads, and/or length of the original nucleic acid molecule in the sample) typically allows for the assignment of a unique identity to a particular molecule. The length, or number of base pairs, of an individual sequence read are also optionally used to assign a unique identity to a given molecule. As described herein, fragments from a single strand of nucleic acid having been assigned a unique identity, may thereby permit subsequent identification of fragments from the parent strand, and/or a complementary strand.

In some embodiments, molecular barcodes are introduced at an expected ratio of a set of identifiers (e.g., a combination of unique or non-unique molecular barcodes) to molecules in a sample. One example format uses from about 2 to about 1,000,000 different molecular barcodes, or from about 5 to about 150 different molecular barcodes, or from about 20 to about 50 different molecular barcodes, ligated to both ends of a target molecule. Alternatively, from about 25 to about 1,000,000 different molecular barcodes may be used. For example, 20-50×20-50 molecular barcodes can be used. In some embodiments, 20-50 different molecular barcodes can be used. In some embodiments, 5-100 different molecular barcodes can be used. In some embodiments, 5-150 molecular barcodes can be used. In some embodiments, 5-200 different molecular barcodes can be used. Such numbers of identifiers are typically sufficient for different molecules having the same start and stop points to have a high probability (e.g., at least 94%, 99.5%, 99.99%, or 99.999%) of receiving different combinations of identifiers. In some embodiments, about 80%, about 90%, about 95%, or about 99% of molecules have the same combinations of molecular barcodes.

In some embodiments, the assignment of unique or non-unique molecular barcodes in reactions is performed using methods and systems described in, e.g., U.S. Pat. Application Nos. 20010053519, 20030152490, and 20110160078, and U.S. Pat. Nos. 6,582,908, 7,537,898, 9,598,731, and 9,902,992, each of which is hereby incorporated by reference in its entirety. Alternatively, in some embodiments, different nucleic acid molecules of a sample may be identified using only endogenous sequence information (e.g., start and/or stop positions, sub-sequences of one or both ends of a sequence, and/or lengths).

### Amplification

Sample nucleic acids flanked by adapters are typically amplified by PCR and other amplification methods using nucleic acid primers binding to primer binding sites in adapters flanking a DNA molecule to be amplified. In some embodiments, amplification methods involve cycles of extension, denaturation and annealing resulting from thermocycling, or can be isothermal as, e.g., in transcription mediated amplification. Other amplification exemplary methods that are optionally utilized, include the ligase chain reaction, strand displacement amplification, nucleic acid sequence-based amplification, and self-sustained sequence-based replication, among other approaches.

One or more rounds of amplification cycles are generally applied to introduce molecular barcodes and/or sample indexes to a nucleic acid molecule using conventional nucleic acid amplification methods. The amplifications are typically conducted in one or more reaction mixtures. Molecular barcodes and sample indexes are optionally introduced simultaneously, or in any sequential order. In some embodiments, molecular barcodes and sample indexes are introduced prior to and/or after sequence capturing operations are performed. In some embodiments, only the molecular barcodes are introduced prior to probe capturing and the sample indexes are introduced after sequence capturing operations are performed. In certain embodiments, both the molecular barcodes and the sample indexes are introduced prior to performing probe-based capturing operations. In some embodiments, the sample indexes are introduced after sequence capturing operations are performed. Typically, sequence capturing protocols involve introducing a single-stranded nucleic acid molecule complementary to a targeted nucleic acid sequence, e.g., a coding sequence of a genomic region and mutation of such region is associated with a cancer type. Alternatively or additionally, typically the amplification reactions generate a plurality of non-uniquely or uniquely tagged nucleic acid amplicons with molecular barcodes and sample indexes at size ranging from about 200 nucleotides (nt) to about 700 nt, from 250 nt to about 350 nt, or from about 320 nt to about 550 nt. In some embodiments, the amplicons have a size of about 300 nt. In some embodiments, the amplicons have a size of about 500 nt.

### Enrichment

Sequences can be enriched prior to sequencing. Enrichment can be performed for specific target regions or nonspecifically (‘target sequences’). In some embodiments, targeted regions of interest may be enriched with capture probes (‘baits’) selected for one or more bait set panels using a differential tiling and capture technique. A differential tiling and capture scheme uses bait sets of different relative concentrations to differentially tile (e.g., at different ‘resolutions’) across genomic regions associated with baits, subject to a set of constraints (e.g., sequencer constraints such as sequencing load, utility of each bait, etc.), and capture them at a desired level for downstream sequencing. These targeted genomic regions of interest may include natural or synthetic nucleotide sequences of the nucleic acid construct. In some embodiments, biotin-labeled beads with probes to one or more regions of interest can be used to capture target sequences, optionally followed by amplification of those regions, to enrich for the regions of interest.

Sequence capture may include the use of oligonucleotide probes that hybridize to the target sequence. A probe set strategy can involve tiling the probes across a region of interest. Such probes can be, e.g., about 60 to 120 bases long. The set can have a depth of about 2×, 3×, 4×, 5×, 6×, 8×, 9×, 10×, 15×, 20×, 50×, or more than 50×. The effectiveness of sequence capture depends, in part, on the length of the sequence in the target molecule that is complementary (or nearly complementary) to the sequence of the probe.

In some embodiments, the plurality of genomic regions includes genetic variants found in the Catalogue of Somatic Mutations in Cancer (COSMIC), The Cancer Genome Atlas (TCGA), or the Exome Aggregation Consortium (ExAC). In some cases, genetic variants may belong to a pre-defined set of clinically actionable variants. For example, such variants may be found in various databases of variants whose presence in a sample of a subject have been shown to correlate with or be indicative of a disease or disorder (e.g., cancer) in the subject. Such databases of variants may include, e.g., COSMIC, TCGA, and the ExAC. A pre-defined set of such catalogued variants may be designated for further bioinformatics analysis due to their relevance to clinical decision-making (e.g., diagnosis, prognosis, treatment selection, targeted treatment, treatment monitoring, monitoring for recurrence, etc.). Such a pre-defined set may be determined based on, e.g., analysis of clinical samples (e.g., of patient cohorts with known presence or absence of a disease or disorder) as well as annotation information from public databases and clinical literature.

### Sequencing

Sample nucleic acids flanked by adapters with or without prior amplification can be subject to sequencing. Sequencing methods include, e.g., Sanger sequencing, high-throughput sequencing, pyrosequencing, sequencing-by-synthesis, single-molecule sequencing, nanopore sequencing, semiconductor sequencing, sequencing-by-ligation, sequencing-by-hybridization, RNA-Seq (from Illumina), Digital Gene Expression (from Helicos BioSciences of Cambridge, Massachusetts), Next generation sequencing, Single Molecule Sequencing by Synthesis or SMSS (from Helicos), massively-parallel sequencing, Clonal Single Molecule Array (from Solexa, a division of Illumina, Inc. of San Diego, California), shotgun sequencing, Ion Torrent, Oxford Nanopore, Roche Genia, Maxim-Gilbert sequencing, primer walking, sequencing using PacBio, SOLiD, Ion Torrent, or Nanopore platforms. Sequencing reactions can be performed in a variety of sample processing units, which may multiple lanes, multiple channels, multiple wells, or other mean of processing multiple sample sets substantially simultaneously. Sample processing unit can also include multiple sample chambers to enable processing of multiple runs simultaneously.

The sequencing reactions can be performed on one or more nucleic acid fragment types or regions known to contain markers of cancer or other diseases. The sequencing reactions can also be performed on any nucleic acid fragment present in the sample. The sequence reactions may be performed on at least about 5%, 10%, 15%, 20%, 25%, 30%, 40%, 50%, 60%, 70%, 80%, 90%, 95%, 99%, 99.9% or 100% of the genome. In other cases, sequence reactions may be performed on less than about 5%, 10%, 15%, 20%, 25%, 30%, 40%, 50%, 60%, 70%, 80%, 90%, 95%, 99%, 99.9% or 100% of the genome.

Simultaneous sequencing reactions may be performed using multiplex sequencing techniques. In some cases, cell free polynucleotides may be sequenced with at least 1000, 2000, 3000, 4000, 5000, 6000, 7000, 8000, 9000, 10000, 50000, or 100,000 sequencing reactions. In other cases, cell free polynucleotides may be sequenced with less than 1000, 2000, 3000, 4000, 5000, 6000, 7000, 8000, 9000, 10000, 50000, or 100,000 sequencing reactions. Sequencing reactions may be performed sequentially or simultaneously. Subsequent data analysis may be performed on all or part of the sequencing reactions. In some cases, data analysis may be performed on at least 1000, 2000, 3000, 4000, 5000, 6000, 7000, 8000, 9000, 10000, 50000, or 100,000 sequencing reactions. In other cases, data analysis may be performed on less than 1000, 2000, 3000, 4000, 5000, 6000, 7000, 8000, 9000, 10000, 50000, or 100,000 sequencing reactions. An exemplary read depth is 1000-50000 reads per locus (base). In some embodiments, read depth can be greater than 50000 reads per locus (base).

### Analysis

Sequencing according to embodiments of the disclosed analysis techniques generates a plurality of sequencing reads or reads. Sequencing reads or reads according to the disclosed analysis techniques generally include sequences of nucleotide data less than about 150 bases in length, or less than about 90 bases in length. In certain embodiments, reads are between about 80 and about 90 bases, e.g., about 85 bases in length. In some embodiments, methods of the disclosed analysis techniques are applied to very short reads, i.e., less than about 50 or about 30 bases in length. Sequencing read data can include the sequence data as well as meta information. Sequence read data can be stored in any suitable file format including, e.g., VCF files, FASTA files or FASTQ files.

FASTA is originally a computer program for searching sequence databases and the name FASTA has come to also refer to a standard file format. See Pearson & Lipman, 1988, “Improved tools for biological sequence comparison,” PNAS 85:2444-2448. A sequence in FASTA format begins with a single-line description, followed by lines of sequence data. The description line is distinguished from the sequence data by a greater-than (‘>’) symbol in the first column. The word following the ‘>’ symbol is the identifier of the sequence, and the rest of the line is the description (both are optional). There should be no space between the ‘>’ and the first letter of the identifier. It is recommended that all lines of text be shorter than 80 characters. The sequence ends if another line starting with a ‘>’ appears; this indicates the start of another sequence.

The FASTQ format is a text-based format for storing both a biological sequence (usually nucleotide sequence) and its corresponding confidence scores. It is similar to the FASTA format but with confidence scores following the sequence data. Both the sequence letter and confidence score are encoded with a single ASCII character for brevity. The FASTQ format is a de facto standard for storing the output of high throughput sequencing instruments such as the Illumina Genome Analyzer, as described by, e.g., Cock et al. (“The Sanger FASTQ file format for sequences with quality scores, and the Solexa/Illumina FASTQ variants,” Nucleic Acids Res 38(6):1767-1771, 2009), which is hereby incorporated by reference in its entirety.

For FASTA and FASTQ files, meta information includes the description line and not the lines of sequence data. In some embodiments, for FASTQ files, the meta information includes the confidence scores. For FASTA and FASTQ files, the sequence data begins after the description line and is present typically using some subset of IUPAC ambiguity codes optionally with ‘-’. In a preferred embodiment, the sequence data will use the A, T, C, G, and N characters, optionally including ‘-’ or U as-needed (e.g., to represent gaps or uracil).

In some embodiments, the at least one master sequence read file and the output file are stored as plain text files (e.g., using encoding such as ASCII; ISO/IEC 646; EBCDIC; UTF-8; or UTF-16). A computer system provided by the disclosed analysis techniques may include a text editor program capable of opening the plain text files. A text editor program may refer to a computer program capable of presenting contents of a text file (such as a plain text file) on a computer screen, allowing a human to edit the text (e.g., using a monitor, keyboard, and mouse) . Exemplary text editors include, without limit, Microsoft Word, emacs, pico, vi, BBEdit, and TextWrangler. Preferably, the text editor program is capable of displaying the plain text files on a computer screen, showing the meta information and the sequence reads in a human-readable format (e.g., not binary encoded but instead using alphanumeric characters as they may be used in print human writing).

While methods have been discussed with reference to FASTA or FASTQ files, methods and systems of the disclosed analysis techniques may be used to compress any suitable sequence file format including, e.g., files in the Variant Call Format (VCF) format. A typical VCF file will include a header section and a data section. The header contains an arbitrary number of meta-information lines, each starting with characters ‘##’, and a TAB delimited field definition line starting with a single ‘#’ character. The field definition line names eight mandatory columns and the body section contains lines of data populating the columns defined by the field definition line. The VCF format is described by Danecek et al. (“The variant call format and VCFtools,” Bioinformatics 27(15):2156-2158, 2011), which is hereby incorporated by reference in its entirety. The header section may be treated as the meta information to write to the compressed files and the data section may be treated as the lines, each of which will be stored in a master file only if unique.

Certain embodiments of the disclosed analysis techniques provide for the assembly of sequencing reads. In assembly by alignment, e.g., the sequencing reads are aligned to each other or aligned to a reference sequence. By aligning each read, in turn to a reference genome, all of the reads are positioned in relationship to each other to create the assembly. In addition, aligning or mapping the sequencing read to a reference sequence can also be used to identify variant sequences within the sequencing read. Identifying variant sequences can be used in combination with the methods and systems described herein to further aid in the diagnosis or prognosis of a disease or condition, or for guiding treatment decisions.

In some embodiments, any or all of the operations are automated. Alternatively, methods of the disclosed analysis techniques may be embodied wholly or partially in one or more dedicated programs, e.g., each optionally written in a compiled language such as C++ then compiled and distributed as a binary. Methods of the disclosed analysis techniques may be implemented wholly or in part as modules within, or by invoking functionality within, existing sequence analysis platforms. In certain embodiments, methods of the disclosed analysis techniques include a number of operations that are all invoked automatically responsive to a single starting cue (e.g., one or a combination of triggering events sourced from human activity, another computer program, or a machine). Thus, the disclosed analysis techniques provide methods in which any or the operations or any combination of the operations can occur automatically responsive to a cue. Automatically generally means without intervening human input, influence, or interaction (i.e., responsive only to original or pre-cue human activity).

The system also encompasses various forms of output, which includes an accurate and sensitive interpretation of the subject nucleic acid. The output of retrieval can be provided in the format of a computer file. In certain embodiments, the output is a FASTA file, FASTQ file, or VCF file. Output may be processed to produce a text file, or an XML file containing sequence data such as a sequence of the nucleic acid aligned to a sequence of the reference genome. In other embodiments, processing yields output containing coordinates or a string describing one or more mutations in the subject nucleic acid relative to the reference genome. Alignment strings may include Simple UnGapped Alignment Report (SUGAR), Verbose Useful Labeled Gapped Alignment Report (VULGAR), and Compact Idiosyncratic Gapped Alignment Report (CIGAR) (Ning et al., Genome Research 11(10):1725-9, 2001, which is hereby incorporated by reference in its entirety). These strings are implemented, e.g., in the Exonerate sequence alignment software from the European Bioinformatics Institute (Hinxton, United Kingdom).

In some embodiments, a sequence alignment is produced (such as, e.g., a sequence alignment map or SAM, or binary alignment map or BAM file) including a CIGAR string (the SAM format is described, e.g., by Li et al., “The Sequence Alignment/Map format and SAMtools,” Bioinformatics, 25(16):2078-9, 2009, which is hereby incorporated by reference in its entirety). In some embodiments, CIGAR displays or includes gapped alignments one-per-line. CIGAR is a compressed pairwise alignment format reported as a CIGAR string. A CIGAR string is useful for representing long (e.g., genomic) pairwise alignments. A CIGAR string is used in SAM format to represent alignments of reads to a reference genome sequence.

A CIGAR string follows an established motif. Each character is preceded by a number, giving the base counts of the event. Characters used can include M, I, D, N, and S (M=match; I=insertion; D=deletion; N=gap; S=substitution). The CIGAR string defines the sequence of matches/mismatches and deletions (or gaps). For example, the CIGAR string 2MD3M2D2M will mean that the alignment contains 2 matches, 1 deletion (number 1 is omitted in order to save some space), 3 matches, 2 deletions and 2 matches.

In some embodiments, a nucleic acid population is prepared for sequencing by enzymatically forming blunt-ends on double-stranded nucleic acids with single-stranded overhangs at one or both ends. In these embodiments, the population is typically treated with an enzyme having a 5′-3′ DNA polymerase activity and a 3′-5′ exonuclease activity in the presence of the nucleotides (e.g., A, C, G and T or U) in the form of dNTPs. Exemplary enzymes or catalytic fragments thereof that are optionally used include Klenow large fragment and T4 polymerase. At 5′ overhangs, the enzyme typically extends the recessed 3′ end on the opposing strand until it is flush with the 5′ end to produce a blunt end. At 3′ overhangs, the enzyme generally digests from the 3′ end up to and sometimes beyond the 5′ end of the opposing strand. If this digestion proceeds beyond the 5′ end of the opposing strand, the gap can be filled in by an enzyme having the same polymerase activity that is used for 5′ overhangs. The formation of blunt-ends on double-stranded nucleic acids facilitates, e.g., the attachment of adapters and subsequent amplification.

In some embodiments, nucleic acid populations are subject to additional processing, such as the conversion of single-stranded nucleic acids to double-stranded and/or conversion of RNA to DNA. These forms of nucleic acid are also optionally linked to adapters and amplified.

With or without prior amplification, nucleic acids subject to the process of forming blunt-ends described above, and optionally other nucleic acids in a sample, can be sequenced to produce sequenced nucleic acids. A sequenced nucleic acid can refer either to the sequence of a nucleic acid (i.e., sequence information) or a nucleic acid whose sequence has been determined. Sequencing can be performed so as to provide sequence data of individual nucleic acid molecules in a sample either directly or indirectly from a consensus sequence of amplification products of an individual nucleic acid molecule in the sample.

In some embodiments, double-stranded nucleic acids with single-stranded overhangs in a sample after blunt-end formation are linked at both ends to adapters including molecular barcodes, and the sequencing determines nucleic acid sequences as well as molecular barcodes introduced by the adapters. The blunt-end DNA molecules are optionally ligated to a blunt end of an at least partially double-stranded adapter (e.g., a Y shaped or bell-shaped adapter). Alternatively, blunt ends of sample nucleic acids and adapters can be tailed with complementary nucleotides to facilitate ligation (for e.g., sticky end ligation).

The nucleic acid sample is typically contacted with a sufficient number of adapters that there is a low probability (e.g., <1 or <0.1%) that any two copies of the same nucleic acid receive the same combination of adapter barcodes (i.e., molecular barcodes) from the adapters linked at both ends. The use of adapters in this manner permits identification of families of nucleic acid sequences with the same start and stop points on a reference nucleic acid and linked to the same combination of molecular barcodes. Such a family represents sequences of amplification products of a nucleic acid in the sample before amplification. The sequences of family members can be compiled to derive consensus nucleotide(s) or a complete consensus sequence for a nucleic acid molecule in the original sample, as modified by blunt end formation and adapter attachment. In other words, the nucleotide occupying a specified position of a nucleic acid in the sample is determined to be the consensus of nucleotides occupying that corresponding position in family member sequences. Families can include sequences of one or both strands of a double-stranded nucleic acid. If members of a family include sequences of both strands from a double-stranded nucleic acid, sequences of one strand are converted to their complement for purposes of compiling all sequences to derive consensus nucleotide(s) or sequences. Some families include only a single member sequence. In this case, this sequence can be taken as the sequence of a nucleic acid in the sample before amplification. Alternatively, families with only a single member sequence can be eliminated from subsequent analysis.

Nucleotide variations in sequenced nucleic acids can be determined by comparing sequenced nucleic acids with a reference sequence. The reference sequence is often a known sequence, e.g., a known whole or partial genome sequence from a subject (e.g., a whole genome sequence of a human subject). The reference sequence can be, e.g., hG19 or hG38. The sequenced nucleic acids can represent sequences determined directly for a nucleic acid in a sample, or a consensus of sequences of amplification products of such a nucleic acid, as described above. A comparison can be performed at one or more designated positions on a reference sequence. A subset of sequenced nucleic acids can be identified including a position corresponding with a designated position of the reference sequence when the respective sequences are maximally aligned. Within such a subset it can be determined which, if any, sequenced nucleic acids include a nucleotide variation at the designated position, and optionally which if any, include a reference nucleotide (i.e., same as in the reference sequence). If the number of sequenced nucleic acids in the subset including a nucleotide variant exceeding a selected threshold, then a variant nucleotide can be called at the designated position. The threshold can be a simple number, such as at least 1, 2, 3, 4, 5, 6, 7, 9, or 10 sequenced nucleic acids within the subset including the nucleotide variant or it can be a ratio, such as a least 0.5, 1, 2, 3, 4, 5, 10, 15, or 20 of sequenced nucleic acids within the subset that include the nucleotide variant, among other possibilities. The comparison can be repeated for any designated position of interest in the reference sequence. Sometimes a comparison can be performed for designated positions occupying at least about 20, 100, 200, or 300 contiguous positions on a reference sequence, e.g., about 20-500, or about 50-300 contiguous positions.

Additional details regarding nucleic acid sequencing, including the formats and applications described herein are also provided in, e.g., Levy et al., Annual Review of Genomics and Human Genetics, 17: 95-115 (2016), Liu et al., J. of Biomedicine and Biotechnology, Volume 2012, Article ID 251364:1-11 (2012), Voelkerding et al., Clinical Chem., 55: 641-658 (2009), MacLean et al., Nature Rev. Microbiol., 7: 287-296 (2009), Astier et al., J Am Chem Soc., 128(5):1705-10 (2006), U.S. Pat. Nos. 6,210,891, 6,258,568, 6,833,246, 7,115,400, 6,969,488, 5,912,148, 6,130,073, 7,169,560, 7,282,337, 7,482,120, 7,501,245, 6,818,395, 6,911,345, 7,501,245, 7,329,492, 7,170,050, 7,302,146, 7,313,308, and 7,476,503, which are each incorporated by reference in their entirety.

### Applications

### Cancer and Other Diseases

Typically, the disease under consideration is a type of cancer. Non-limiting examples of such cancers include biliary tract cancer, bladder cancer, transitional cell carcinoma, urothelial carcinoma, brain cancer, gliomas, astrocytomas, breast carcinoma, metaplastic carcinoma, cervical cancer, cervical squamous cell carcinoma, rectal cancer, colorectal carcinoma, colon cancer, hereditary nonpolyposis colorectal cancer, colorectal adenocarcinomas, gastrointestinal stromal tumors (GISTs), endometrial carcinoma, endometrial stromal sarcomas, esophageal cancer, esophageal squamous cell carcinoma, esophageal adenocarcinoma, ocular melanoma, uveal melanoma, gallbladder carcinomas, gallbladder adenocarcinoma, renal cell carcinoma, clear cell renal cell carcinoma, transitional cell carcinoma, urothelial carcinomas, Wilms tumor, leukemia, acute lymphocytic leukemia (ALL), acute myeloid leukemia (AML), chronic lymphocytic leukemia (CLL), chronic myeloid leukemia (CML), chronic myelomonocytic leukemia (CMML), liver cancer, liver carcinoma, hepatoma, hepatocellular carcinoma, cholangiocarcinoma, hepatoblastoma, Lung cancer, non-small cell lung cancer (NSCLC), mesothelioma, B-cell lymphomas, non-Hodgkin lymphoma, diffuse large B-cell lymphoma, Mantle cell lymphoma, T cell lymphomas, non-Hodgkin lymphoma, precursor T-lymphoblastic lymphoma/leukemia, peripheral T cell lymphomas, multiple myeloma, nasopharyngeal carcinoma (NPC), neuroblastoma, oropharyngeal cancer, oral cavity squamous cell carcinomas, osteosarcoma, ovarian carcinoma, pancreatic cancer, pancreatic ductal adenocarcinoma, pseudopapillary neoplasms, acinar cell carcinomas, Prostate cancer, prostate adenocarcinoma, skin cancer, melanoma, malignant melanoma, cutaneous melanoma, small intestine carcinomas, stomach cancer, gastric carcinoma, gastrointestinal stromal tumor (GIST), uterine cancer, or uterine sarcoma.

Non-limiting examples of other genetic-based diseases, disorders, or conditions that are optionally evaluated using the methods and systems disclosed herein include achondroplasia, alpha-1 antitrypsin deficiency, antiphospholipid syndrome, autism, autosomal dominant polycystic kidney disease, Charcot-Marie-Tooth (CMT), cri du chat, Crohn’s disease, cystic fibrosis, Dercum disease, down syndrome, Duane syndrome, Duchenne muscular dystrophy, Factor V Leiden thrombophilia, familial hypercholesterolemia, familial mediterranean fever, fragile X syndrome, Gaucher disease, hemochromatosis, hemophilia, holoprosencephaly, Huntington’s disease, Klinefelter syndrome, Marfan syndrome, myotonic dystrophy, neurofibromatosis, Noonan syndrome, osteogenesis imperfecta, Parkinson’s disease, phenylketonuria, Poland anomaly, porphyria, progeria, retinitis pigmentosa, severe combined immunodeficiency (scid), sickle cell disease, spinal muscular atrophy, Tay-Sachs, thalassemia, trimethylaminuria, Turner syndrome, velocardiofacial syndrome, WAGR syndrome, Wilson disease, or the like.

Furthermore, in some embodiments, the analysis techniques may be used to assist in the treatment of a type of cancer. Identifying and removing strand bias can improve tissue biopsies to correctly diagnose and administer a patient and identify adequate treatment to treat the patient’s specific genomic lesions.

These methods and provided herein provide a deeper understanding of the changes in DNA and proteins that cause cancer, allowing the identification of biomarkers and design of treatments that target these proteins. Such treatments may include small-molecule drugs or monoclonal antibodies. The methods may also improve biomarker testing in individuals suffering from disease and help determine if the individual is a candidate for a certain drug or combination of drugs based on the presence or absence of the biomarker. Additionally, the methods can improve identification of mutations that contribute to the development of resistance to targeted therapy. Consequently, the analysis techniques may reduce unnecessary or untimely therapeutic interventions, patient suffering, and patient mortality.

Therapies can function by helping the immune system destroy cancer cells. For example, certain targeted therapies may mark cancer cells for the immune system to destroy them. Other targeted therapies may support the immune system to work more effectively against cancer. Yet other therapies may stop cancer cells from growing, for example, by interfering with cancer cell surface markers preventing them from dividing. Additionally, therapies can inhibit signals that promote angiogenesis. Such angiogenesis inhibitors prevent blood supply into the tumor thereby, preventing tumor growth. Other targeted therapies can deliver toxic substances to the tumor. Examples include monoclonal antibodies combined with toxins, chemotherapy, or radiation. Some targeted therapies induce apoptosis or deplete cancer of hormones.

In some embodiments, the therapies are PARP inhibitors such as Olaparib (Lynparza), Rucaparib (Rubraca), Niraparib (Zejula), and Talazoparib (Talzenna). These may be used for treating mutations in BRCA1, BRCA2, ATM, BARD1, BRIP1, CDK12, CHEK1, CHEK2, FANCL, PALB2, RAD51B,RAD51 C, RAD51D and RAD54L alterations, and/or for Homologous Recombination Repair (HRR) genes.

In some embodiments the treatment comprises immunotherapies and/or immune checkpoint inhibitors (ICIS) such as anti-pd-1/pd-11 therapies including pembrolizumab (Keytruda), nivolumab (Opdivo), and cemiplimab (Libtayo), atezolizumab (Tecentriq), durvalumab (Imfinzi), and avelumab (Bavencio). This therapies may be used to treat patients identified as having high microsatellite instability (MSI) status or high tumor mutational burden (TMB).

In some embodiments the therapies target mutated forms of the EGFR protein. Such therapies can include osimertinib (Tagrisso), erlotinib (Tarceva), and gefinitib (Iressa).

Therapies can include one or more of treatments for target therapies, including abemaciclib (Verzenio), abiraterone acetate (Zytiga), acalabrutinib (Calquence), adagrasib (Krazati), ado-trastuzumab emtansine (Kadcyla), afatinib dimaleate (Gilotrif), alectinib (Alecensa), alemtuzumab (Campath), alitretinoin (Panretin), alpelisib (Piqray), amivantamab-vmjw (Rybrevant), anastrozole (Arimidex), apalutamide (Erleada), asciminib hydrochloride (Scemblix), atezolizumab (Tecentriq), atezolizumab (Tecentriq), avapritinib (Ayvakit), avelumab (Bavencio), axicabtagene ciloleucel (Yescarta), axitinib (Inlyta), belinostat (Beleodaq), belzutifan (Welireg), bevacizumab (Avastin), bexarotene (Targretin), binimetinib (Mektovi), blinatumomab (Blincyto), bortezomib (Velcade), bosutinib (Bosulif), brentuximab vedotin (Adcetris), brexucabtagene autoleucel (Tecartus), brigatinib (Alunbrig), cabazitaxel (Jevtana), cabozantinib-s-malate (Cabometyx), cabozantinib-s-malate (Cometriq), capmatinib hydrochloride (Tabrecta), carfilzomib (Kyprolis), cemiplimab-rwlc (Libtayo), ceritinib (Zykadia), cetuximab (Erbitux), ciltacabtagene autoleucel (Carvykti), cobimetinib fumarate (Cotellic), copanlisib hydrochloride (Aliqopa), crizotinib (Xalkori), dabrafenib (Tafinlar), dabrafenib mesylate (Tafinlar), dacomitinib (Vizimpro), daratumumab (Darzalex), daratumumab and hyaluronidase-fihj (Darzalex Faspro), darolutamide (Nubeqa), dasatinib (Sprycel), denileukin diftitox (Ontak), denosumab (Xgeva), dinutuximab (Unituxin), dostarlimab-gxly (Jemperli), durvalumab (Imfinzi), duvelisib (Copiktra), elacestrant dihydrochloride (Orserdu), elotuzumab (Empliciti), enasidenib mesylate (Idhifa), encorafenib (Braftovi), enfortumab vedotin-ejfv (Padcev), entrectinib (Rozlytrek), enzalutamide (Xtandi), erdafitinib (Balversa), erlotinib hydrochloride (Tarceva), everolimus (Afinitor), exemestane (Aromasin), fam-trastuzumab deruxtecan-nxki (Enhertu), fam-trastuzumab deruxtecan-nxki (Enhertu), fedratinib hydrochloride (Inrebic), fulvestrant (Faslodex), futibatinib (Lytgobi), gefitinib (Iressa), gemtuzumab ozogamicin (Mylotarg), gilteritinib fumarate (Xospata), glasdegib maleate (Daurismo), ibritumomab tiuxetan (Zevalin), ibrutinib (Imbruvica), idecabtagene vicleucel (Abecma), idelalisib (Zydelig), imatinib mesylate (Gleevec), infigratinib phosphate (Truseltiq), inotuzumab ozogamicin (Besponsa), iobenguane I 131 (Azedra), ipilimumab (Yervoy), isatuximab-irfc (Sarclisa), ivosidenib (Tibsovo), ixazomib citrate (Ninlaro), lanreotide acetate (SomatulineDepot), lapatinib ditosylate (Tykerb), larotrectinib sulfate (Vitrakvi), lenvatinib mesylate (Lenvima), letrozole (Femara), lisocabtagene maraleucel (Breyanzi), loncastuximab tesirine-lpyl (Zynlonta), lorlatinib (Lorbrena), lutetium Lu 177 vipivotide tetraxetan (Pluvicto), lutetium Lu 177-dotatate (Lutathera), margetuximab-cmkb (Margenza), midostaurin (Rydapt), mirvetuximab soravtansine-gynx (Elahere), mobocertinib succinate (Exkivity), mogamulizumab-kpkc (Poteligeo), mosunetuzumab-axgb (Lunsumio), moxetumomab pasudotox-tdfk(Lumoxiti), naxitamab-gqgk (Danyelza), necitumumab (Portrazza), neratinib maleate (Nerlynx), nilotinib (Tasigna), niraparib tosylate monohydrate (Zejula), nivolumab (Opdivo), nivolumab and relatlimab-rmbw (Opdualag), obinutuzumab (Gazyva), ofatumumab (Arzerra), olaparib (Lynparza), olutasidenib (Rezlidhia), osimertinib mesylate (Tagrisso), pacritinib citrate (Vonjo), palbociclib (Ibrance), panitumumab (Vectibix), pazopanib hydrochloride(Votrient), pembrolizumab (Keytruda), pemigatinib(Pemazyre), pertuzumab (Perjeta), pertuzumab, trastuzumab, and hyaluronidase-zzxf (Phesgo), pexidartinib hydrochloride (Turalio), pirtobrutinib (Jaypirca), polatuzumab vedotin-piiq (Polivy), ponatinib hydrochloride (Iclusig), pralatrexate (Folotyn), pralsetinib (Gavreto), radium 223 dichloride (Xofigo), ramucirumab (Cyramza), regorafenib (Stivarga), retifanlimab-dlwr (Zynyz), ribociclib (Kisqali), ripretinib (Qinlock), rituximab (Rituxan), rituximab and hyaluronidase human (Rituxan Hycela), romidepsin (Istodax), rucaparib camsylate(Rubraca), ruxolitinib phosphate (Jakafi), sacituzumab govitecan-hziy (Trodelvy), selinexor (Xpovio), selpercatinib (Retevmo), selumetinib sulfate (Koselugo), siltuximab (Sylvant), sirolimus protein-bound particles (Fyarro), sonidegib (Odomzo), sorafenib tosylate (Nexavar), sotorasib (Lumakras), sunitinib malate (Sutent), tafasitamab-cxix (Monjuvi), tagraxofusp-erzs (Elzonris), talazoparib tosylate (Talzenna), tamoxifen citrate (Soltamox), tazemetostat hydrobromide (Tazverik), tebentafusp-tebn (Kimmtrak), teclistamab-cqyv (Tecvayli), temsirolimus (Torisel), tepotinib hydrochloride (Tepmetko), tisagenlecleucel (Kymriah), tisotumab vedotin-tftv (Tivdak), tivozanib hydrochloride (Fotivda), toremifene (Fareston), trametinib (Mekinist), trametinib dimethyl sulfoxide (Mekinist), trastuzumab (Herceptin), tremelimumab-actl (Imjudo), tretinoin (Vesanoid), tucatinib (Tukysa), vandetanib (Caprelsa), vemurafenib (Zelboraf), venetoclax (Venclexta), vismodegib (Erivedge), vorinostat (Zolinza), zanubrutinib (Brukinsa), ziv-aflibercept (Zaltrap).

The methods disclosed herein are practical in analyzing sequencing reads derived from tumor samples to detect somatic mutations. By filtering out false positive variants which result from tissue processing and/or storage, the method improves the specificity to detect true cancer-causing mutations. Accurate detection of true cancer-causing mutations is critical in precision medicine since these mutations may inform treatment selection, assessment of minimal residual disease, and resistance. For example, DNA damage due to tissue storage/processing is a stochastic process where mutations can occur anywhere in the genome including biomarker genes such as EGFR, ALK, KRAS, p53, BRCA1, and BRCA2. Unless effectively filtered, these mutations will be called, potentially leading to incorrect treatment selection and disease prognosis. For example, a mutation in BRCA½ in a breast cancer patient may determine treatment course (such as with a PARP inhibitor), prognosis, and whether a double mastectomy is recommended. Furthermore, removal of false positive variants and accurate variant calling enables identification of cancer biomarkers and treatment selection, for example an accurately called EGFR mutation (e.g., T790M substitution, exon 19 deletion, exon 21 L858R substitution, exon 20 instertion mutations) may be effectively targeted using osimertinib (Tagrisso), erlotinib (Tarceva), and gefinitib (Iressa).

### EXAMPLE

### Example 1: Determining a Dynamic Confidence Metric According to an Embodiment of the Disclosure

For a tissue sample at Chromosome 2, location 29449762, there may be T-to-C SNV having a Watson reference allele of 647 (or a Watson strand having 647 molecules for a reference allele), a Crick reference allele of 665 (or a Crick strand having 665 molecules for the reference allele), a Watson alternate allele of 2 (or the Watson strand having 2 molecules for the alternate allele) and a Crick alternate allele of 1 (or the Crick alternate allele having 1 molecule of the alternate allele). For this SNV, the odds ratio is

$$\frac{647 ⋅ 1}{665 ⋅ 2} ,$$

the second or inverse odds ratio is

$$\frac{665 ⋅ 2}{647 ⋅ 1} ,$$

the reference ratio is

$$\frac{647}{665} ,$$

the alternate ratio is

$$\frac{1}{2} ,$$

and the symmetric normalized odds ratio is

$$\text{ln} \left(\left(\frac{647 ⋅ 1}{665 ⋅ 2}\right) + \left(\frac{665 ⋅ 2}{647 ⋅ 1}\right)\right) + ln \left(\frac{647}{665}\right) - ln \left(\frac{1}{2}\right) \text{or 1} \text{.5987} \text{.}$$

Note that the use of the phrases ‘capable of,’ ‘capable to,’ ‘operable to,’ or ‘configured to’ in one or more embodiments, refers to some apparatus, logic, hardware, and/or element designed in such a way to enable use of the apparatus, logic, hardware, and/or element in a specified manner.

In the preceding description, we refer to ‘some embodiments’. Note that ‘some embodiments’ describes a subset of all of the possible embodiments, but does not always specify the same subset of embodiments. Moreover, note that the numerical values provided are intended as illustrations of the analysis techniques. In other embodiments, the numerical values can be modified or changed.

Moreover, as sequencing and biopsy assays are changed (e.g., in sequencing depth and panels of common SNPs), methods and systems of the present disclosure may be modified as needed to obtain a set of applicable threshold values (e.g., one or more criteria/threshold to determine a dynamic confidence metric of a sample).

The foregoing description is intended to enable any person skilled in the art to make and use the disclosure, and is provided in the context of a particular application and its requirements. Moreover, the foregoing descriptions of embodiments of the present disclosure have been presented for purposes of illustration and description only. They are not intended to be exhaustive or to limit the present disclosure to the forms disclosed. Accordingly, many modifications and variations will be apparent to practitioners skilled in the art, and the general principles defined herein may be applied to other embodiments and applications without departing from the spirit and scope of the present disclosure. Additionally, the discussion of the preceding embodiments is not intended to limit the present disclosure. Thus, the present disclosure is not intended to be limited to the embodiments shown, but is to be accorded the widest scope consistent with the principles and features disclosed herein.

What is claimed is:

1. A computer system, comprising:
an interface circuit;
a computation device coupled to the interface circuit; and
memory, coupled to the computation device, configured to store program instructions, wherein, when executed by the computation device, the program instructions cause the computer system to perform one or more operations comprising:
receiving information corresponding to identified molecules of deoxyribonucleic acid (DNA) from a tissue sample;
determining a symmetric normalized odds ratio based at least in part on the information, wherein the symmetric normalized odds ratio corresponds to damage of the DNA and determining the symmetric normalized odds ratio comprises:
computing a first odds ratio;
computing a second odds ratio, wherein a numerator and a denominator in the second odds ratio are reversed relative to the first odds ratio;
summing the first odds ratio and the second odds ratio; and
normalizing the summation; and
calculating a confidence metric of one or more of the molecules based at least in part on the symmetric normalized odds ratio and a threshold, wherein the confidence metric corresponds to a probability that the one or more molecules are identified correctly.

2. The computer system of claim 1, wherein the DNA damage is associated with formalin fixing and paraffin embedding of the tissue sample.

3. The computer system of claim 1, wherein the DNA damage comprises oxidated degradation of guanine to 8-oxoguanine (oxoG) or formaldehyde-induced DNA and chromatin damage; and
wherein the formaldehyde-induced DNA and chromatin damage comprises: deamination, depurination, or histone-DNA crosslinks.

4. The computer system of claim 1, wherein the information comprises DNA sequences that each correspond to a single strand of DNA from the tissue sample; and
wherein the DNA damage is associated with strand bias.

5. The computer system of claim 1, wherein the operations comprise calling variants in the DNA based at least in part on the confidence metric.

6. The computer system of claim 5, wherein the operations comprise filtering out a subset of the call variants based at least in part on the confidence metric.

7. The computer system of claim 6, wherein the subset comprises false-positive variant calls in the call variants associated with the DNA damage or that are incorrectly labeled as contamination.

8. The computer system of claim 6, wherein the subset comprise the variant calls associated with strand bias.

9. The computer system of claim 5, wherein the variant calls single-nucleotide variants (SNVs).

10. The computer system of claim 1, wherein the operations comprise adjusting one or more sonication parameters for subsequent sonication of the tissue sample based at least in part on the confidence metric.

11. The computer system of claim 10, wherein the confidence metric corresponds to a level of DNA fragmentation.

12. The computer system of claim 1, wherein a given odds ratio in the first odds ratio and the second odds ratio is computed based at least in part on: a number of occurrences of a first allele on a first strand in the DNA; a number of occurrences of the first allele on a second strand in the DNA; a number of occurrences of a second allele on the first strand in the DNA; and a number of occurrences of the second allele on the second strand in the DNA.

13. The computer system of claim 12, wherein the first allele has a majority allele frequency and the second allele has a minority allele frequency.

14. The computer system of claim 1, wherein the one or more operations comprise determining a quality metric of the tissue sample by aggregating multiple confidence metrics for the molecules in the tissue sample.

15. A non-transitory computer-readable storage medium for use in conjunction with a computer system, the computer-readable storage medium configured to store program instructions that, when executed by the computer system, causes the computer system to perform one or more operations comprising:
receiving information corresponding to identified molecules of deoxyribonucleic acid (DNA) from a tissue sample;
determining a symmetric normalized odds ratio based at least in part on the information, wherein the symmetric normalized odds ratio corresponds to damage of the DNA and determining the symmetric normalized odds ratio comprises:
computing a first odds ratio;
computing a second odds ratio, wherein a numerator and a denominator in the second odds ratio are reversed relative to the first odds ratio;
summing the first odds ratio and the second odds ratio; and
normalizing the summation; and
calculating a confidence metric of one or more of the molecules based at least in part on the symmetric normalized odds ratio and a threshold, wherein the confidence metric corresponds to a probability that the one or more molecules are identified correctly.

16. The non-transitory computer-readable storage medium of claim 15, wherein the information comprises DNA sequences that each correspond to a single strand of DNA from the tissue sample; and
wherein the DNA damage is associated with strand bias.

17. The non-transitory computer-readable storage medium of claim 15, wherein the operations comprise: calling variants in the DNA based at least in part on the confidence metric; or adjusting one or more sonication parameters for subsequent sonication of the tissue sample based at least in part on the confidence metric.

18. A method for detecting damage of deoxyribonucleic acid (DNA) from a tissue sample, comprising:
by a computer system:
receiving information corresponding to identified molecules of deoxyribonucleic acid (DNA) in the tissue sample;
determining a symmetric normalized odds ratio based at least in part on the information, wherein the symmetric normalized odds ratio corresponds to damage of the DNA and determining the symmetric normalized odds ratio comprises:
computing a first odds ratio;
computing a second odds ratio, wherein a numerator and a denominator in the second odds ratio are reversed relative to the first odds ratio;
summing the first odds ratio and the second odds ratio; and
normalizing the summation; and
calculating a confidence metric of one or more of the molecules based at least in part on the symmetric normalized odds ratio and a threshold, wherein the confidence metric corresponds to a probability that the one or more molecules are identified correctly.

19. The method of claim 18, wherein the information comprises DNA sequences that each correspond to a single strand of DNA from the tissue sample; and
wherein the DNA damage is associated with strand bias.

20. The method of claim 18, wherein the method comprises calling variants in the DNA based at least in part on the confidence metric.
